(19) United States
(12) Patent Application Publication
(10) Pub. No.: US 2023/0014065 A1
(43) Pub. Date: Jan. 19, 2023

(54) SYSTEM AND METHOD FOR MAPPING MUSCULAR ACTIVATION

(71) Applicants: X-trodes LTD, Herzeliya (IL); Ramot at Tel-Aviv University Ltd., Tel-Aviv (IL)

(72) Inventors: Yael HANEIN, Tel-Aviv (IL); Stas STEINBERG, Tel-Aviv (IL); Lilah INZELBERG YIFA, Tel-Aviv (IL)

(73) Assignees: X-trodes LTD, Herzeliya (IL); Ramot at Tel-Aviv University Ltd., Tel-Aviv (IL)

(21) Appl. No.: 17/952,387

(22) Filed: Sep. 26, 2022

Related U.S. Application Data
(63) Continuation of application No. PCT/IL2021/050360, filed on Mar. 30, 2021.
(60) Provisional application No. 63/001,589, filed on Mar. 30, 2020.

(51) Int. Cl. A61B 5/389; A61B 5/257; A61B 5/296; A61B 5/00

(52) U.S. Cl. CPC A61B 5/389; A61B 5/257; A61B 5/296; A61B 5/746; A61B 5/743; A61B 2505/09; A61B 2562/046

(57) ABSTRACT

A system for determining muscle activation comprises a set of electrode adherable to a skin of a subject, and a processor in communication with the electrodes. The processor has a circuit configured for receiving locations of the electrodes and electrical signals detected by the electrodes, analyzing the signals to identify a section of an active muscle, identifying locations of at least a segment of active muscles and activation patterns of the active muscles based on the identified section, and constructing a displayable map of the locations and the activation patterns, wherein patterns corresponding to different active muscles are distinguishable on the map.

### RELATED APPLICATIONS

This application is a Continuation of PCT Patent Application No. PCT/IL2021/050360 having International filing date of Mar. 30, 2021, which claims the benefit of priority under 35 USC § 119(e) of U.S. Provisional Patent Application No. 63/001,589 filed on Mar. 30, 2020. The contents of the above applications are all incorporated by reference as if fully set forth herein in their entirety.

### FIELD AND BACKGROUND OF THE INVENTION

The present invention, in some embodiments thereof, relates to a non-invasive monitoring and, more particularly, but not exclusively, to a system and method for mapping muscular activation.

Human facial muscle activation underlies highly sophisticated signaling mechanisms that are important for healthy physiological function. Current technologies for analyzing facial muscle activity are based on electromyography electrodes, which are usually in the form of stiff metal pads. Also known is the use of gel for improving the electrical communication between the skin and the electrodes.

### SUMMARY OF THE INVENTION

The Inventors of the present invention realized the need to analyze muscle activation (for example, facial muscles, diaphragm muscle, limb muscles) at high-resolution and in a non-invasive manner for the diagnosis and treatment of many medical, psychological and cognitive conditions as well of for cosmetic purposes. The Inventors found that current clinical examination methods are neither precise nor quantitative. For example, stiff metal pads lack flexibility and thus suffer from poor adhesion to the skin, resulting in low signal-to-noise ratio, especially during muscle activation, and gelled electrodes are usually bulky and cumbersome and suffer from reduced signal over time due to gel dehydration. The inventors realize that visual inspection is highly subjective and builds on highly trained personnel, and that video processing lacks physiological validity. The inventors found that both visual inspection and video processing are insensitive to isometric muscle activations since in some cases the muscles can be activated, without a noticeably change in their length.

According to an aspect of some embodiments of the present invention there is provided a system for determining muscle activation. The system comprises a set of electrode adherable to a skin of a subject, and a processor in communication with the electrodes. The processor has a circuit configured for receiving locations of the electrodes and electrical signals detected by the electrodes, analyzing the signals to identify a section of an active muscle, identifying locations of at least a segment of active muscles and activation patterns of the active muscles based on the identified section, and constructing a displayable map of the locations and the activation patterns, wherein patterns corresponding to different active muscles are distinguishable on the map.

According to some embodiments of the invention the map overlays an image of a body portion and/or a graphical representation of the electrodes.

According to some embodiments of the invention the analysis is carried out by a blind source separation algorithm.

According to some embodiments of the invention the circuit is configured for detecting muscle unit action potential (MUAP) activity based on an output of the blind source separation algorithm.

According to some embodiments of the invention the set of electrodes comprises two subsets of electrode for receiving signals from respective two opposite sides of a portion of the skin.

According to some embodiments of the invention the set of electrodes comprises two subsets of electrode for receiving signals from respective two limbs.

According to some embodiments of the invention the circuit is configured to access a database storing a library of activation patterns and associated control commands, to search the database for a database activation pattern matching the identified activation pattern, and to extract from the library control commands associated with the matched database activation pattern.

According to some embodiments of the invention the circuit is configured to transmit the extracted control commands to an appliance.

According to some embodiments of the invention the circuit is configured for at least one member of a group consisting of: determining muscle fatigue, performance training, for rehabilitation, for determining muscle pain and any combination thereof.

According to some embodiments of the invention the circuit is configured to generate a warning if a parameter is outside at least one predetermined limit.

According to some embodiments of the invention the parameter comprises at least one of level of pain, force exerted by a muscle, level of muscle fatigue, and asymmetry of muscle activity.

According to some embodiments of the invention the warning is provided by a member of a group consisting of visually, audibly or tactilely and any combination thereof.

According to some embodiments of the invention the system is in use in relation to plastic surgery, for a member of a group consisting of improvement of facial symmetry, during rehabilitation physiotherapy and any combination thereof.

According to some embodiments of the invention the system is in use for neurorehabilitation.

According to some embodiments of the invention the circuit is configured for at least one of: providing characterization of walking, providing assessment of post-stroke recovery, providing assessment of post-spinal cord injury motor recovery, providing spasticity assessment, providing biofeedback, employing serious games, providing indication of muscle synergies, controlling a prosthesis, controlling an exoskeleton, controlling a robot.

According to some embodiments of the invention the system is in use for at least one of: extracting neural control strategies, myoelectric manifestations of muscle fatigue, and myoelectric manifestations of cramps.

According to some embodiments of the invention the circuit is configured for identifying the locations and the activation patterns, while the subject is moving.

According to some embodiments of the invention the circuit is configured for identifying the locations and the activation patterns, while the active muscles do not change their length or shape.

According to an aspect of some embodiments of the present invention there is provided a method of determining muscle activation. The method comprises adhering a set of electrodes to a skin of a subject, receiving locations of the electrodes and electrical signals detected by the electrodes, analyzing the signals to identify a section of an active muscle, identifying locations of at least segments of active muscles and activation patterns of the active muscles based on the identified section, and constructing a displayable map of the locations and the activation patterns, wherein patterns corresponding to different active muscles are distinguishable on the map. Various operations of the method are optionally and preferably carried out by a processor.

According to some embodiments of the invention the map overlays an image of a body portion and/or a graphical representation of the electrodes.

According to some embodiments of the invention the body portion is selected from a group consisting of a portion of a face, a portion of a neck, a portion of an arm, a portion of a leg, a portion of a hand, a portion of a foot, a portion of a torso, a portion of a head, and any combination thereof.

According to some embodiments of the invention the analysis is carried out by a blind source separation algorithm.

According to some embodiments of the invention the blind source separation algorithm comprises an algorithm selected from a group consisting of independent component analysis (ICA), fast independent component analysis (fastICA), principal component analysis, singular value decomposition, dependent component analysis, non-negative matrix factorization, low-complexity coding and decoding, stationary subspace analysis, common spatial pattern analysis and any combination thereof.

According to some embodiments of the invention the method comprises detecting muscle unit action potential (MUAP) activity based on an output of the blind source separation algorithm.

According to some embodiments of the invention the adhering comprises adhering two subsets of electrode to respective two opposite sides of a portion of the skin.

According to some embodiments of the invention the adhering comprises adhering two subsets of electrode to respective two limbs.

According to some embodiments of the invention the method comprises accessing a database storing a library of activation patterns and associated control commands, search the database for a database activation pattern matching the identified activation pattern, and extracting from the library control commands associated with the matched database activation pattern.

According to some embodiments of the invention the method comprises transmitting the extracted control commands to an appliance.

According to some embodiments of the invention the appliance comprises at least one of a robot and a personal mobile device.

According to some embodiments of the invention the method is in use for at least one of: determining muscle fatigue, performance training, for rehabilitation, for determining muscle pain and any combination thereof.

According to some embodiments of the invention the method comprises generating a warning if a parameter is outside at least one predetermined limit. According to some embodiments of the invention the parameter comprises at least one of: level of pain, force exerted by a muscle, level of muscle fatigue, and asymmetry of muscle activity.

According to some embodiments of the invention the method is in use in relation to plastic surgery, for a member of a group consisting of improvement of facial symmetry, during rehabilitation physiotherapy and any combination thereof.

According to some embodiments of the invention the method is in use for neurorehabilitation.

According to some embodiments of the invention the method comprises at least one of: providing characterization of walking, providing assessment of post-stroke recovery, providing assessment of post-spinal cord injury motor recovery, providing spasticity assessment, providing biofeedback, employing serious games, providing indication of muscle synergies, controlling a prosthesis, controlling an exoskeleton, controlling a robot.

According to some embodiments of the invention the method is in use for at least one of: extracting neural control strategies, myoelectric manifestations of muscle fatigue, and myoelectric manifestations of cramps.

According to some embodiments of the invention the identifying the locations and the activation patterns is executed while the subject is moving.

According to some embodiments of the invention the identifying the locations and the activation patterns is executed while the active muscles do not change their length or shape.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 illustrates the muscles of the face;

FIGS. 2A-B show electrodes in contact with a skin;

FIG. 3 shows an upper surface of a hemi-facial electrode array according to some embodiments of the present invention;

FIG. 4 shows a lower surface of a hemi-facial electrode array according to some embodiments of the present invention;

FIG. 5 shows an embodiment of an electrode array in position on the face;

FIG. 6 shows typical results for facial expressions, obtained in experiments performed according to some embodiments of the present invention;

FIGS. 7A-D are images obtained in experiments performed according to some embodiments of the present invention, showing facial expressions;

FIGS. 8A-D shows independent component (IC) maps of muscle activation patterns obtained in experiments performed according to some embodiments of the present invention overplaying face images;

FIG. 9A shows normalized un-mixing matrix weights at each electrode, obtained in experiments performed according to some embodiments of the present invention;

FIG. 9B shows distance from cluster centroids, obtained in experiments performed according to some embodiments of the present invention;

FIG. 9C schematically illustrates a spatial representation of the IC sources as a contour form on an electrode array layout, according to some embodiments of the present invention;

FIG. 9D schematically illustrates contours of cluster centroids on an array layout, according to some embodiments of the present invention;

FIGS. 10A-D show distinct groups of derived clusters (FIGS. 10A and 10C), and respective IC maps (FIGS. 10B and 10D), obtained in experiments performed according to some embodiments of the present invention;

FIG. 11A shows facial building blocks (FBBs) on a hemi-facial electrode array layout, according to some embodiments of the present invention;

FIG. 11B shows the FBBs of FIG. 11A on a 3D model of a head;

FIGS. 12A and 12B show spontaneous IC maps overlaid on lateral images of a face (FIG. 12A) and in relation to an electrode array (FIG. 12B), obtained in experiments performed according to some embodiments of the present invention;

FIGS. 13A-D show normalized histograms of FBBs for four individuals, obtained in experiments performed according to some embodiments of the present invention;

FIGS. 14A-D show a summary of all normalized FBBs distributions per category, obtained in experiments performed according to some embodiments of the present invention;

FIG. 15A illustrates a flexor digitorum muscle;

FIG. 15B is an image showing electrodes placed on a forearm, according to some embodiments of the present invention;

FIG. 16 shows typical results recorded according to some embodiments of the present invention by electrodes placed on a forearm;

FIGS. 17A-B show independent components of a contracting flexor digitorum muscle at different levels of exerted force;

FIG. 18A shows two components with Motor Unit Action Potential (MUAP) pulse trains while performing flexion under 0.5N force, obtained in experiments performed according to some embodiments of the present invention;

FIG. 18B shows a linear relationship between pulse frequency (MUAP Rate (MR)) and force, obtained in experiments performed according to some embodiments of the present invention;

FIGS. 19A-B shows separation of components from noisy data, according to some embodiments of the present invention;

FIG. 20 shows activation of the flexor muscles for finger flexion, as obtained in experiments performed according to some embodiments of the present invention;

FIGS. 21A-C shows independent components extracted, during experiments performed according to some embodiments of the present invention, from different flexion tasks as normalized weights distributed over 16 electrodes and sorted into 10 clusters using cosine distance k-means.

FIGS. 22A-C shows the cosine similarity between the components in FIGS. 21A-C, according to some embodiments of the present invention.

FIGS. 23A and 23B show two repeating IC's over six repetitions for a task which utilizes the palmaris longus muscle, as obtained in experiments performed according to some embodiments of the present invention;

FIGS. 24A-D show six repetitions of an activity, obtained in experiments performed according to some embodiments of the present invention;

FIG. 25 is a diagram schematically illustrating an embodiment of a system for use in determination of breathing or heart function, according to some embodiments of the present invention;

FIG. 26 is an image showing an exemplary embodiment of measurement of the sEMG signal in place on the chest employed in experiments performed according to some embodiments of the present invention;

FIGS. 27A and 27B show raw and smoothed sEMG signal, obtained in experiments performed according to some embodiments of the present invention; and

FIGS. 28A and 28B show an exemplary embodiment of measurement of Electrocardiography (ECG), with FIG. 28A showing a device in position according to some embodiments of the present invention on a chest, and FIG. 28B showing an ECG signal a recorded according to some embodiments of the present invention from a single channel of the device.

### DESCRIPTION OF SPECIFIC EMBODIMENTS OF THE INVENTION

The present invention, in some embodiments thereof, relates to a non-invasive monitoring and, more particularly, but not exclusively, to a system and method for mapping muscular activation.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present embodiments comprise a set of electrodes configured to provide a high-definition map of muscle activation in a region below the skin. The present embodiments can also comprise a circuit configured to execute program instructions that analyze muscle activation so as to determine which muscles are activated and how strong the activation is. The set of electrodes are non-invasively attachable to the region of skin, imposing no mechanical disturbance to the user and are configured to be customized for the user.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pulls these data structures or values for use in subsequent operation. All these operations are well-known to those skilled in the art of computer systems.

Processing operations described herein may be performed by means of processor circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

Fields in which such the map of the present embodiments can be used include, but are not limited to, medicine, esthetic treatments, and sport. Objective quantification of the muscular activity signatures holds exciting opportunities in many fields such as diagnostic pathology, prosthetics control, rehabilitation after stroke or injury, sports and entertainment, neurological and psychological evaluation, respiratory monitoring.

The electrodes measure electrical activity in different parts of the region of skin under examination. From patterns of electrical activity, the system of the present embodiments can be determined muscle or muscle-segment locations, muscle coordination. The system can optionally and preferably also determine whether a muscle or one or more muscle groups are non-functional, ill-functioning or improperly functioning. The system can also be used to induce functionality in non-functioning muscles or improve functionality in ill-functioning or improperly-functioning muscles or muscle groups.

The set of electrodes of the present embodiments optionally and preferably comprise a wearable customizable high-resolution surface electromyography electrode array.

The wearable high-resolution surface electromyography electrode array of the present embodiments are optionally and preferably printed electrodes, such as, but not limited to, printed carbon electrodes. Other conductive printed electrodes are also contemplated. The electrodes are optionally and preferably deposited, more preferably printed, on a substrate characterized by a Young's modulus of less than 30 MPa, e.g., from about 1 MPa to about 30 MPa. A representative example of a material suitable for use as a substrate is, without limitation a polyurethane. The diameter of the electrodes comprises is typically from about 3 mm to about 10 mm. The inventors found that such dimensions allow high density, while maintaining low noise levels and good conformity with the skin. The thickness of the substrate is typically from about 60 to about 150 μm, e.g., 80 μm.

As used herein the term “about” refers to ±10%

In some embodiments of the present invention the signals from the electrodes are analyzed to provide a map of muscle activation patterns and locations of active muscles or segments of active muscles. The maps can be derived from repeated voluntary muscle activations. An independent component (IC) analysis procedure and a machine learning procedure can then identify activation patterns that are subject-specific, and optionally and preferably also activation patterns that are universal or specific to a group of subjects.

The application of IC analysis and machine learning procedure of the present embodiments to data acquired by the electrodes is advantageous because it allows identifying the locations of the active muscles (or of their segments) and the activation patterns, even when the subject is moving. This is unlike conventional techniques in which the subject is restricted to be static. An additional advantage is that it allows the identifying of activation patterns of active muscles while the muscles do not change their length or shape (neither during the contraction of the muscle nor during the return of the muscle to its relaxed state).

The activation patterns can optionally and preferably be used to identify normal and abnormal activation patterns and, therefore, normal and abnormal patterns of use of the muscles. In some embodiments, the patterns can be used as input to a training program to improve muscle use, as an identifier of tiredness in muscles, muscle sections or muscle groups, as an identifier of overuse of muscles or muscle groups, and any combination thereof.

In some embodiments, the set of electrodes are attached to the face of the subject.

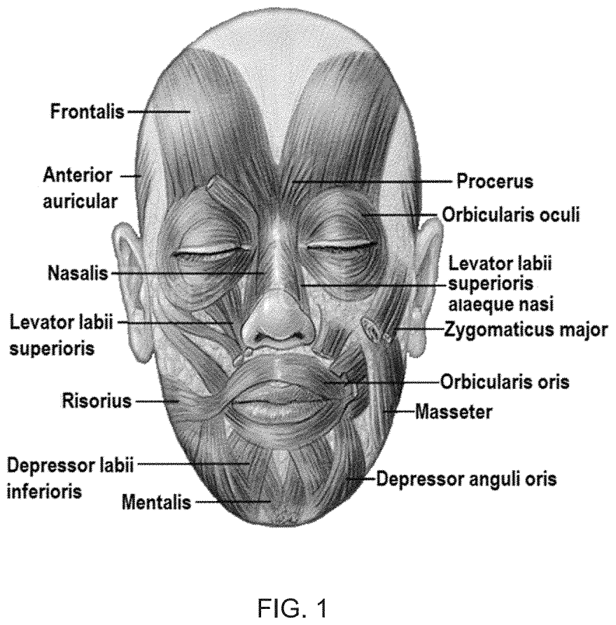

Human facial muscle is illustrated in FIG. 1. Activation of human facial muscle underlies highly sophisticated signaling mechanisms that are believed to be important for healthy physiological function. Accordingly, analysis of facial muscle activation at a high resolution and in a non-invasive manner according to some embodiments of the present invention can aid in diagnosing and treating many medical conditions.

In preferred embodiments, the electrode array comprises printed dry electrodes having multiple recording sites that allow a customized match to human anatomy with synchronous recordings from numerous muscles using a single electrode array. For example, in experiments performed according to some embodiments of the present invention, a hemi-facial 16 electrode array, which covers many lateral parts of the face, has been employed, allowing mapping several facial expressions.

Electrical activity of a muscle can be found by employing a technique that can sense a change in bioelectrical potential which can be picked-up from the surface of the skin. Examples include, but are not limited to, electroencephalography (EEG), electrocardiography (ECG), Electrooculography (EOG) (recording eye movement), electro-olfactography (EOLG), and electromyography (EMG). In some preferred embodiments, at least one of EMG, EoG and ECG is used.

Muscle activation maps can be derived from repeated voluntary muscle activations. The IC analysis and machine learning procedure of the present embodiments can identify consistent building block activation patterns within and between participants. A further analysis of spontaneous muscle activations (e.g., smiles or other expressions, in case of facial muscles) can be used to classify muscle activation sources. This can optionally and preferably be used to extract consistent subject-specific activation, and also estimate inter-subject variability. The analysis can also be used to classify muscle activation sources for, for non-limiting example, expressions such as commanded smiling, spontaneous frowning and commanded frowning.

The present embodiments allow automated and objective mapping of, for example, facial expressions in general and in the assessment of normal and abnormal smiling in particular and, for another non-limiting example, muscle use in the forearm. Other parts of the body for which muscle activation can be mapped can include the upper arm, a leg, a torso, and the neck. The system can also be used on animals, for non-limiting example, on domestic pets, farm animals, guard animals and racing animals.

Other applications can include detecting use of illegal drugs, and detecting bombs and explosives. For example, the system of the present embodiments can be used during training of animals to detect bombs and explosives, and/or to identify muscular activity of the animal upon sensing existence of a bomb or explosive.

The electrode array optionally and preferably, together with the aforementioned IC analysis and machine learning procedure, establish a non-invasive and high-resolution approach to specific muscle detection and identification at the individual level. For example, once robust normal activation facial building blocks (FBBs) have been characterized, spontaneous, natural, and even clinical events can benefit from such findings. This can in turn be used in diagnostic, assessment and therapeutic purposes, as well as in human-machine-interface.

Unlike conventional techniques, the system and method of the present embodiments does not need to use visual methodologies, such as imaging, that require proper lighting and resolution. Thus, in various exemplary embodiments of the invention the system and method identify activation patterns of one or more active muscles without analyzing optical signals received from the body. This is advantageous because it does not demand a camera view of the subject under investigation. For example, when facial muscle activation patterns are desired, there is no need for a frontal full-face view of the face of the subject.

The system and method of the present embodiments can achieve deep and detailed muscle resolution that cannot be achieved by image analysis. This high-resolution capacity can provide, for non-limiting example, valuable information regarding synergetic muscle activity and precise identification of muscle sections.

Hereinbelow, a representative example is shown of the synergy of two spatially separable regions, such as smiling activating the Zygomaticus major (lower face) and Glabellar (Corrugator supercilii and Procerus) muscles (upper face) simultaneously. This observation is in line with the dual nerve supply from the frontal as well as the zygomatic and buccal branches of the facial nerve. After receiving innervation from the zygomatic branch, the buccal branch forms the angular nerve to supply the Glabellar muscles [Caminer D M, Newman M I and Boyd J B, Angular nerve: New insights on innervation of the corrugator supercilii and procerus muscles. J. Plast. Reconstr. Aesthetic Surg. 59 366-72, 2006; Yu M and Wang S-M, Anatomy, Head and Neck, Eye Corrugator Muscle. (StatPearls Publishing), 2019]. The far reaching application of such outcome can be used for accurate aesthetic and reconstructive surgery, e.g. denervation of Glabellar muscles from fibers of the frontal branch has shown low success and unpredictable results since the innervation is also supplied by the buccal and zygomatic branches. High-resolution neurophysiological preoperative evaluation in laboratory or natural setting may lead to better surgical decisions.

The system optionally and preferably comprises an electrode array to capture sEMG data. The sEMG data are transferred, preferably wirelessly, but possibly wiredly, to a processor having a circuit configured to run dedicated software. The processor can be local, can be remote and can be in the cloud. The software comprises an algorithmic solution to cluster the independent sEMG sources and to derive therefrom individual mappings for each participant. The individual mappings can be combined to identify robust building blocks (RBBs) which are associated with specific muscles. The patterns of RBB use can then determine patterns of use for muscle groups, individual muscles and portions of muscles for different types of activity utilizing the muscle groups, individual muscles and portions of muscles. For non-limiting example, RBB patterns can be used to distinguish between different facial expressions and even between different types of smiles. In other non-limiting examples, RBB patterns can distinguish between types of muscle use to move different fingers, and can be used to identify changes in muscle activation patterns in response to a mechanical load experienced by the respective muscle or muscles; and the RBBs at the diaphragm region can be used to distinguish between different types of breathing, for use as a diagnostic in determining onset of breathing difficulties and onset of increased severity of respiratory-related illnesses.

The IC procedure used by the technique of the present embodiments typically executes a blind source separation algorithm, such as, but not limited to, independent component analysis (ICA), fast independent component analysis (fastICA), principal component analysis, singular value decomposition, dependent component analysis, non-negative matrix factorization, low-complexity coding and decoding, stationary subspace analysis, common spatial pattern analysis and any combination thereof.

For use on the face, the IC procedure disclosed herein can capture activation of specific muscles in voluntary and spontaneous expressions.

When used on the face, the system of the present embodiments can comprise a hemi-facial electrode array over at least a portion of the upper and lower parts of the face. For use on the face, the IC procedure can extract derived data for each participant of a research group separately. Then, all individual mappings can be combined to identify robust FBBs which are associated with specific facial muscles. From these, a classification approach can determine FBB activation during spontaneous smiles.

Proper facial musculature activation is advantageous both for physiological needs (e.g. swallowing, chewing, speaking, eating or closing the eyes) and for social interactions (e.g. smiling, frowning). Many clinical disorders are manifested by abnormal facial activation patterns leading to physiological and psycho-social burden. In Parkinson's disease, for example, hypomimia; the reduction of spontaneous facial expression is a major challenge with severe esthetic and psychological ramifications [Argaud S, Delplanque S, Houvenaghel J-F, Auffret M, Duprez J, Vérin M, Grandjean D and Sauleau P, Does Facial Amimia Impact the Recognition of Facial Emotions? An EMG Study in Parkinson's Disease. ed S Kotz, PLoS One 11 e0160329, 2016; Bologna M, Berardelli I, Paparella G, Marsili L, Ricciardi L, Fabbrini G and Berardelli, A, Altered Kinematics of Facial Emotion Expression and Emotion Recognition Deficits Are Unrelated in Parkinson's Disease. Front. Neurol. 7 1-7, 2016]. Tourette syndrome, an opposite example, is typified by fast and repetitive involuntary facial movements in the form of tics [Brandt V C, Patalay P, Bäumer T, Brass M and Münchau A, Tics as a model of over-learned behavior-imitation and inhibition of facial tics. Mov. Disord. 31 1155-62, 2016; Muth C C, Tics and Tourette Syndrome. JAMA 317 1592, 2017]. Uncontrolled facial episodes of laughter or crying occur in amyotrophic lateral sclerosis [Thakore N J and Pioro E P, Laughter, crying and sadness in ALS. J. Neurol. Neurosurg. Psychiatry 88 825-31, 2017]. Abnormal facial muscle activation patterns appear in many other conditions such as hemifacial spasm (HFS), facial paresis, aberrant regeneration and synkinesis [Valls-Solé J and Montero J, Movement disorders in patients with peripheral facial palsy, Mov. Disord. 18 1424-35, 2003; Wang A and Jankovic J, Hemifacial spasm: Clinical findings and treatment. Muscle Nerve 21 1740-7, 1998; Yaltho T C and Jankovic J, The many faces of hemifacial spasm: Differential diagnosis of unilateral facial spasms. Mov. Disord. 26 1582-92, 2011]. In HFS, for example, involuntary and irregular movements occur in muscles innervated by the seventh cranial nerve [Yaltho]. Typical symptoms include “twitching” of the lower eyelid, followed by spasms of other facial muscles [Wang]. Damaged facial muscle activation may also be the result of cancer or trauma. Following tumor removal surgery, muscle activation may be damaged resulting in deterioration of speech, deglutition and dryness of the eyes [Shah J P and Gil Z, Current concepts in management of oral cancer—Surgery. Oral Oncol. 45 394-401, 2009; Eskes M, van Alphen M J A, Smeele L E, Brandsma D, Balm A J M, van der Heijden F, Alphen M J A Van and Smeele L E, Predicting 3D lip movement using facial sEMG: a first step towards estimating functional and aesthetic outcome of oral cancer surgery. Med. Biol. Eng. Comput. 55 573-83, 2017]. Facial plastic surgery and nerve grafting are challenged by the complex anatomy of facial musculature and nerve anatomy, especially in reanimation procedures such as smile reconstruction [Fattah A, Borschel G H, Manktelow R T, Bezuhly M and Zuker R M, Facial Palsy and Reconstruction. Plast. Reconstr. Surg. 129 340e-352e, 2012; Manktelow R T, Tomat L R, Zuker R M and Chang M, Smile Reconstruction in Adults with Free Muscle Transfer Innervated by the Masseter Motor Nerve: Effectiveness and Cerebral Adaptation. Plast. Reconstr. Surg. 118 885-99; 2006; Guntinas-Lichius O, Genther D J and Byrne P J, Facial Reconstruction and Rehabilitation. Advances in Oto-Rhino-Laryngology vol 78 pp 120-31, 2016].

In some embodiments, facial muscle activation during smiling is analyzed. It is appreciated that although smiling is ubiquitous, understanding its spatial structure and function is advantageous and is useful in psychological and neurological evaluation. Extensive investigations have demonstrated the importance and complexity of smiles in countless fields, ranging from human emotion perception and action, behavioral aspects, well-being, human-robot communication, security, lie detection and aesthetics to pathological manifestations [Ugail H and Aldahoud A A A, Computational Techniques for Human Smile Analysis. (Cham: Springer International Publishing, 2019); Ekman P, Telling lies. (New York-London: W. W. Norton & Company, 1985); Abel E L and Kruger M L, Smile Intensity in Photographs Predicts Longevity. Psychol. Sci. 21 542-4, 2010; Kraus M W and Chen T-W D, A winning smile? Smile intensity, physical dominance, and fighter performance. Emotion 13 270-9, 2013].

The Inventors found that conventional methods for facial muscle activation mapping are neither precise nor quantitative. A widely used method is the facial action coding system (FACS). FACS is based on observed displacements of facial features [Ekman P and Friesen W V., Measuring facial movement. Environ. Psychol. Nonverbal Behav. 1 56-75, 1976] and is used extensively in psychological and behavioral studies. Conventionally, FACS requires a trained human coder and lengthy analysis. Computational approaches, where image or video analysis are employed, have partially automated the process, yet require a vast amount of data to account for facial rotation, translation and scale invariance relative to camera location. Proper visual path and illumination are needed to reach high accuracy [Ugail; Barrett L F, Adolphs R, Marsella S, Martinez A M and Pollak S D, Emotional Expressions Reconsidered: Challenges to Inferring Emotion From Human Facial Movements. Psychol. Sci. Public Interes. 20 1-68, 2019].

Several computerized systems have been developed to measure facial movements by analyzing facial reflecting dots in optical motion systems [Hontanilla B and Aubá C, Automatic three-dimensional quantitative analysis for evaluation of facial movement. J. Plast. Reconstr. Aesthetic Surg. 61 18-30, 2008; Coulson S E, Croxson G R and Gilleard W L, Quantification of the Three-Dimensional Displacement of Normal Facial Movement. Ann. Otol. Rhinol. Laryngol. 109 478-83, 2000; Dusseldorp J R, van Veen M M, Mohan S and Hadlock T A, Outcome Tracking in Facial Palsy. Otolaryngol. Clin. North Am. 51 1033-50, 2018]. However, the Inventors found that these techniques lack muscle specificity.

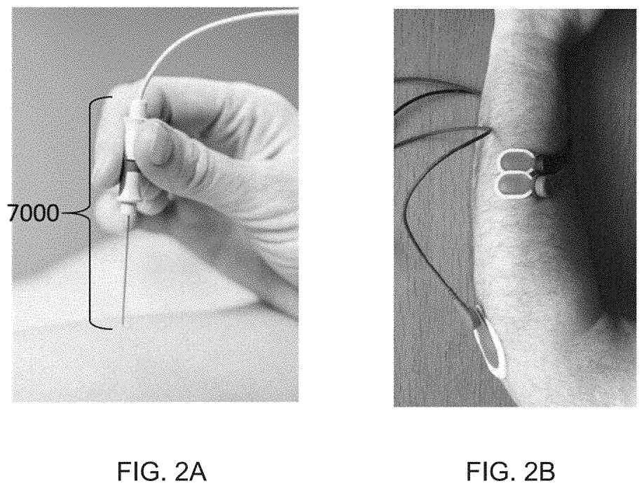

Conventional EMG is known to elucidate muscle activation processes. FIGS. 2A-B are images describing traditional EMG techniques. Needle-EMG is recognized as the gold standard in diagnosis of patterns such as in facial paresis and detection of synkinetic muscle contractions [Valls-Solé; Schumann N P, Bongers K, Guntinas-Lichius O and Scholle H C, Facial muscle activation patterns in healthy male humans: A multi-channel surface EMG study. J. Neurosci. Methods 187 120-8, 2010; Hatem J, Sindou M and Vial C, Intraoperative monitoring of facial EMG responses during microvascular decompression for hemifacial spasm. Prognostic value for long-term outcome: a study in a 33-patient series. Br. J. Neurosurg. 15 496-9, 2001; Drost G, Stegeman D F, van Engelen B G M and Zwarts M J, Clinical applications of high-density surface EMG: A systematic review. J. Electromyogr. Kinesiol. 16 586-602, 2006]. However, needle-EMG is an invasive method that may cause discomfort, pain and even local bleeding, especially in a delicate area such as the face. It is nearly impossible to perform needle-EMG studies in the pediatric population. Surface EMG (sEMG) is a non-invasive alternative to needle-EMG for facial muscle activation analysis, but it is generally limited by low resolution and strong cross-talk [Hug F and Tucker K, Surface Electromyography to Study Muscle Coordination Handbook of Human Motion. ed B Müller, S I Wolf, G-P Brueggemann, Z Deng, A McIntosh, F Miller and W S Selbie (Cham: Springer International Publishing) pp 1-21, 2016]. Both the conventional needle-EMG and the conventional sEMG necessitate artificial settings.

The system of the present embodiments provides a high-resolution sEMG that can be integrated easily with the body portion and be used, for non-limiting example, on the face, the arm, the leg, and the torso.

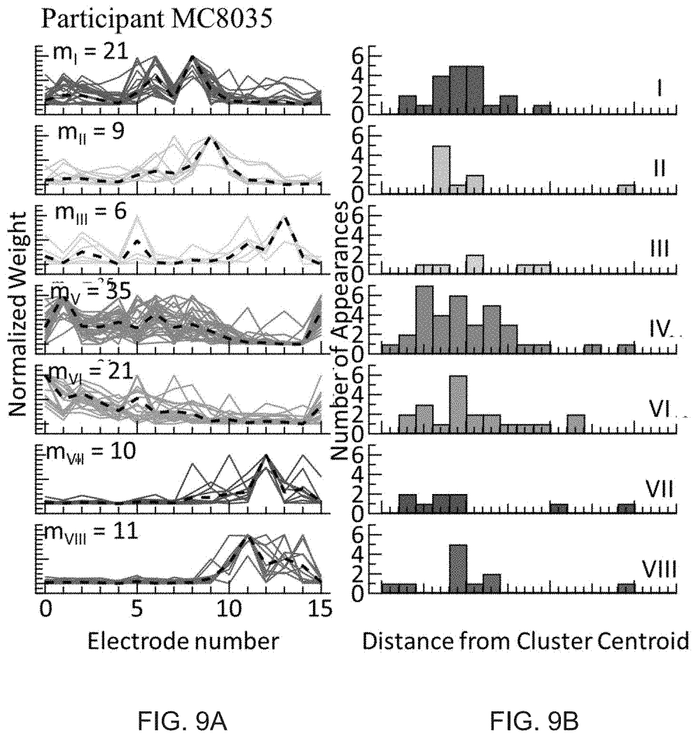

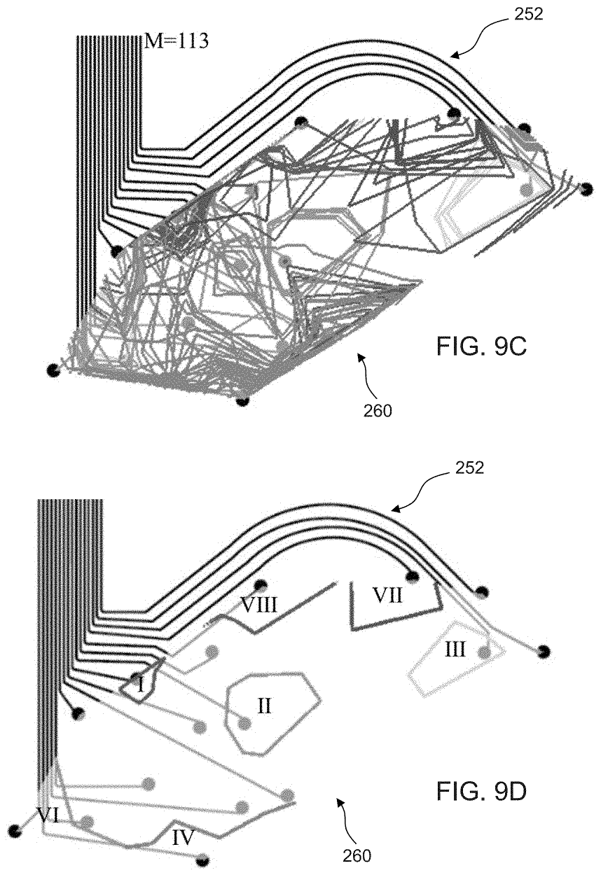

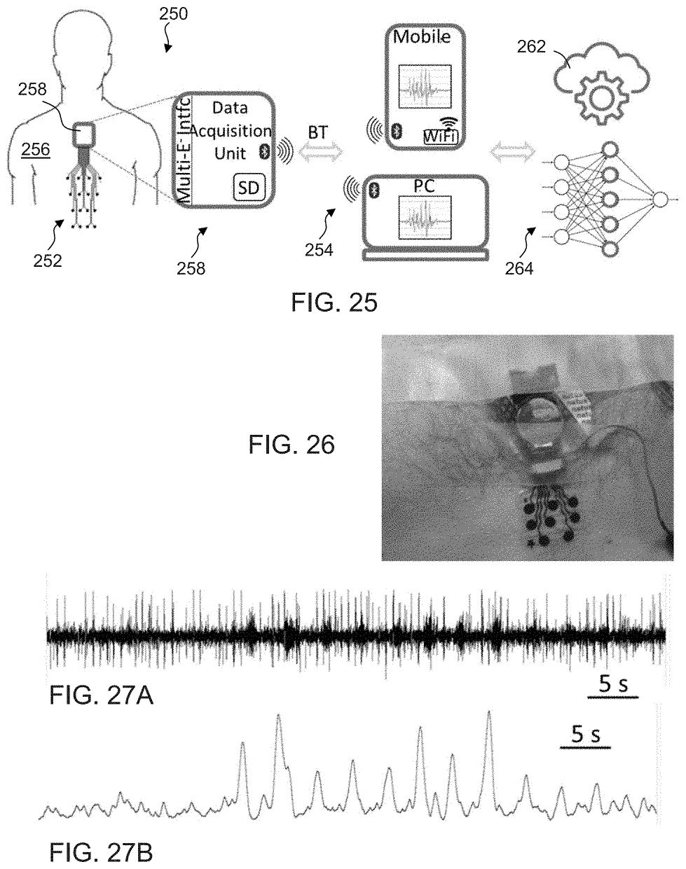

FIG. 25 is a schematic illustration of a system 250 for determining muscle activation, according to some embodiments of the present invention. Shown are set 252 of electrodes adherable to the skin of a subject 256, and a processor 254 in communication with the electrodes 252, and having a circuit configured for receiving electrical signals detected by at least a few of electrodes 252, analyzing the signals to identify at least a section of an active muscle, and identifying, based on the section of the active muscle, a location of the active muscle or a segment of the active muscle, as well as activation patterns of the active muscle. In some embodiments of the present invention processor 254 constructs a displayable map 260 (not shown, see, for example, FIGS. 8A-D, and 9C-D) of the locations and activation patterns, wherein patterns corresponding to different active muscles are distinguishable on map 260. Map 260 can be displayed to overlay an image of a body portion (see, for example, FIGS. 8A-D) and/or a graphical representation of electrodes 252 (see, e.g., FIGS. 9A-C).

In some embodiments of the present invention the electrodes are in the formed of a patch which can be plugged into a miniature wireless Data Acquisition Unit (DAU) 258 that amplifies, digitizes, and transmits the signal using a standard wireless transmission protocol, such as, but not limited to, a Bluetooth protocol. The data can be displayed and stored on a computer 254 or a mobile device using dedicated software. The computer 254 or mobile device is preferably local to the DAU 258 but can be remote from the DAU 258. Data can also be stored in a cloud 262. The analysis can optionally and preferably include application of a machine learning procedure 264. Data analysis can be performed locally or in a cloud-based engine. The results can then be sent in real-time to a physician or health care provider for further evaluation and treatment.

Embodiments of the present invention can provide a means of recording biopotentials of the respiratory muscles (sEMGdi) and the heart (ECG) at a site remote from a clinician or medical setup, such as, but not limited to, a home or a quarantine facility using a proprietary disposable, dry, and flexible multi-electrode array patch applied to the chest region.

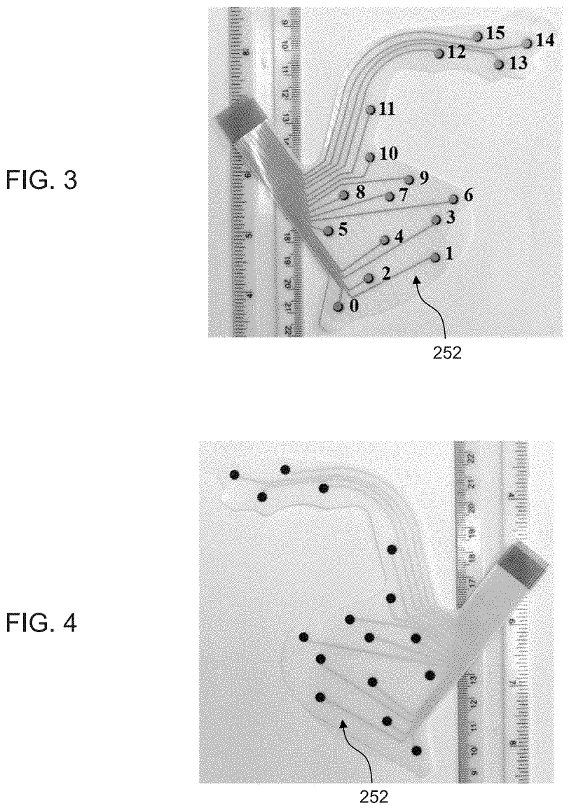

In some embodiments of analysis of facial activation, two hemi-facial electrode arrays are used, one for each side on the face. FIGS. 3 and 4 show, respectively, the upper surface and lower surface of an embodiment of a hemi-facial high-density 16 electrode array. In FIG. 3, electrode numbers are shown. In the embodiment as shown, the array is about 4 mm in diameter; arrays can vary from 1 mm diameter (for pediatric use) to 10 mm diameter. The arrays were screen-printed on a thin and flexible polyurethane substrate with a two-step process using Silver (Ag) and Carbon (C) inks at Tel Aviv University. Packaging was completed at Pronat Industries Ltd. In this embodiment, the lines are silver and the electrodes, carbon.

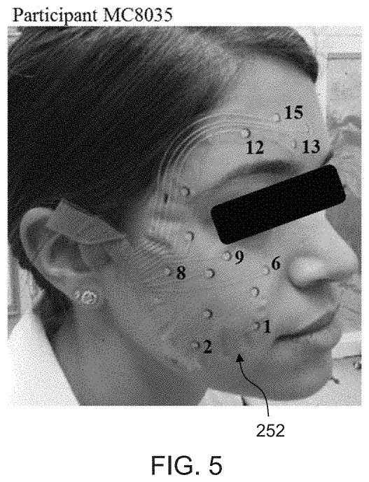

As shown in FIG. 5, in preferred embodiments, the 16 electrode array is configured to cover the jaw, cheek, eye and eyebrow regions while allowing a user to perform natural head and facial movements. For the embodiment shown, electrodes 0-2 are configured to be located near the upper part of the jaw, 3-8 cover the cheek region, 9-11 surround the eye and 12-15 are located above the eyebrow. Other arrangements of the electrodes are also contemplated.

In an embodiment of analysis of sEMG signals, given a set of sEMG signals (observations) \vec{x}₁(t), \vec{x}₂(t), . . . , \vec{x}ₙ(t), where t is the time and n is the number of electrodes, it can be assumed that they are generated as a linear mixture of independent components

$$( \begin{matrix}{\vec{x}}_{1} ( t ) \\ ⋮ \\ {\vec{x}}_{n} ( t )\end{matrix} ) = A ( \begin{matrix}{\vec{s}}_{1} ( t ) \\ ⋮ \\ {\vec{s}}_{1} ( t )\end{matrix} ) \qquad ( 1 )$$

where A is a mixing matrix and \vec{s}₁(t), \vec{s}₂(t), . . . , \vec{s}ₙ(t) are the original signals, as generated by the muscles. A is a square matrix of size n×n. A blind source separation algorithm such as a fastICA algorithm can be applied to find the original signals, \vec{s}ᵢ(t), i=1, . . . , n, from the mixed observations \vec{n}ᵢ(t), i=1, . . . , n:

$$( \begin{matrix}{\vec{s}}_{1} ( t ) \\ ⋮ \\ {\vec{s}}_{n} ( t )\end{matrix} ) = W ( \begin{matrix}{\vec{x}}_{1} ( t ) \\ ⋮ \\ {\vec{x}}_{n} ( t )\end{matrix} ) \qquad ( 2 )$$

where W=A⁻¹ is the un-mixing matrix of size n×n. Therefore, the muscle activity signals \vec{s}₁(t), \vec{s}₂(t), . . . , \vec{s}ₙ(t) can be identified from the set of sEMG signals \vec{x}₁(t), \vec{x}₂(t), . . . , \vec{x}ₙ (t) and their weight in each electrode. In an embodiment, the fastICA algorithm, using a MATLAB 2.5 package [Hyvarinen 1: Hyvarinen A, Karhunen J and Erkki O, Independent Component Analysis. (John Wiley & Sons), 2001], can be applied with a nonlinear fit for facial mapping. The nonlinear fit can be, for example, a polynomial fit. In experiments performed by the inventors a 3rd degree polynomial was employed, but other packages and/or other nonlinearity functions can be used for facial mapping, limb muscle mapping and torso muscle mapping.

For facial mapping, the electrode location and inverse un-mixing matrix, W, can be used to generate IC patterns for each facial calibration expression in each repetition separately. The machine learning (e.g., clustering) procedure can then be applied to classify the IC patterns and to construct a map which is specific to a activation of a particular muscle, or a combined maps in which activations of different muscles are distinguishable. In preferred embodiments, the fastICA needs not to be limited in the number of extracted output components, but will result in a number of components consistent with the number of electrodes. For the device as tested in the examples herein, 16 components were found, consistent with the 16 electrodes.

In some embodiments of the present invention the data processing flow is as follows. The signals from the electrodes are digitized to provide multidimensional sEMG data. The data are filtered, for example, with a notch or comb filter of about 50 Hz and a bandpass filter. Typically, the pass band is from about 5 to about 1000 Hz, more preferably from about 20 Hz to from about 500 Hz, but other bands are also contemplated. The bandpass filter is preferably applied to include physiologically relevant data and to reject low-frequency and high frequency noise. The sEMG sources are optionally and preferably calculated by applying blind source separation (e.g., fastICA) to the data. This provides a plurality of data components, one data component for each sEMG source.

The data components are optionally and preferably represented as digital vectors. The components can then be classified by applying a machine learning procedure, such as, but not limited to, a clustering procedure, to the components. In experiments performed by the inventors k-means clustering was employed. The K-means procedure employs a successive sequence of iterations so as to minimize a predetermined criterion, such as the sum of the squares of the distances from all the data points in the cluster to their nearest cluster centers. The k-means procedure is advantageous because the number of clusters can be determined a priori thereby reducing the complexity of the procedure. In some embodiments of the present invention the k-means procedure is executed for total of from about 5 to about 15 clusters. Other clustering procedures (hierarchical or partitional) such as, but not limited to, graph a clustering procedure which is based on graph theory, scale-space clustering, hard or fuzzy C-means clustering, minimal spanning tree clustering, and a clustering procedure which is based on Potts-spins, are also contemplated.

In various exemplary embodiments of the invention the clustering is according to the temporal and spectral signal properties of the components. The classified components can then be mapped spatially using the electrode positions as landmarks. In some embodiments of the present invention the centroids of the clusters are spatially resolved over the locations of the contacts of the electrodes. The cluster centroids are referred to as FBBs. Preferably, a map is constructed by marking activation patterns around the spatially resolved centroids. Typically, the patterns include contours defined at locations at which the muscle activations reach maxima within a predetermined tolerance (e.g., tolerance of from about 0.5 to about 3 standard deviations).

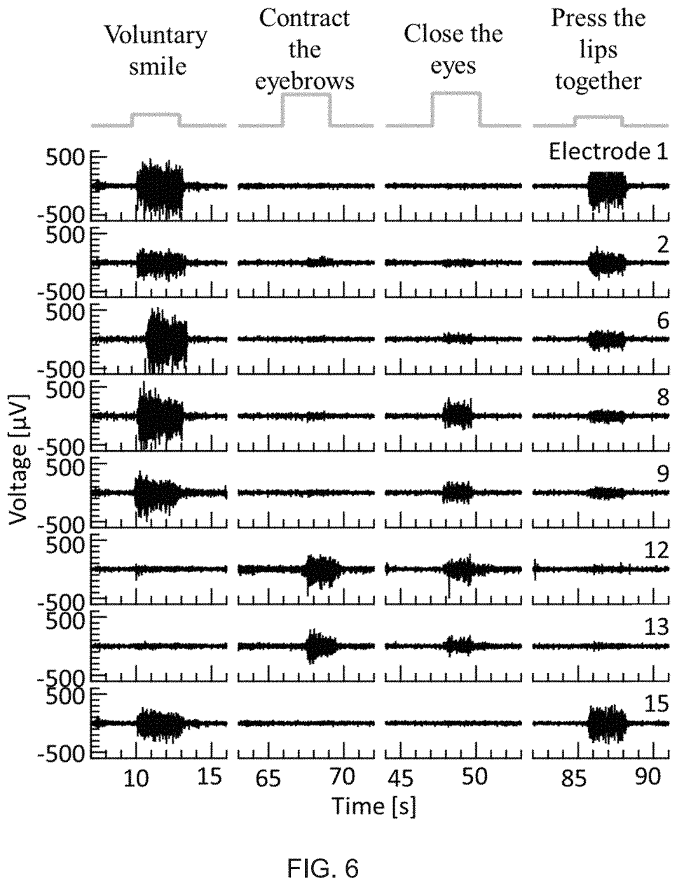

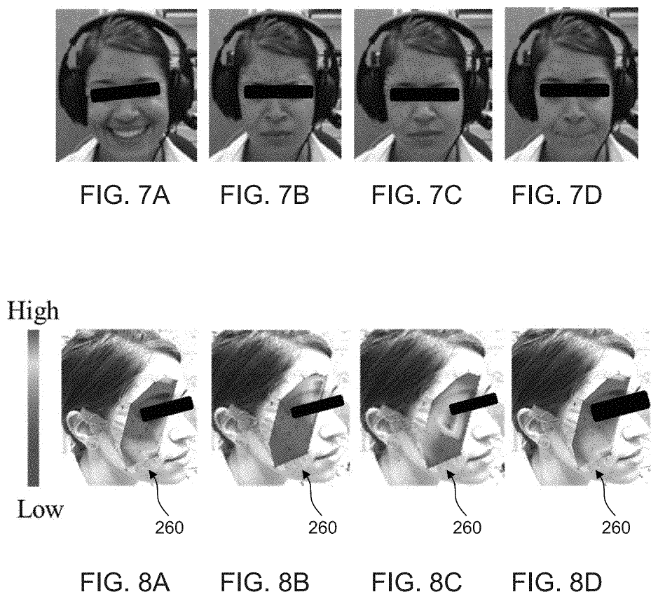

Typical results for facial activation are shown in the Examples section that follows, see FIG. 6 for facial expressions such as those shown in FIG. 7A-D.

FIGS. 8A-D the Examples section that follows show IC maps of muscle activation patterns, interpolated to the lateral photographs of each subject and color-coded (red denotes highest muscle activation and blue the lowest). The IC contours were spatially defined by the maximal muscle activation location at the IC map minus 1.5 standard deviations from that maximum.

The un-mixing matrix, W, can change its column order every time the fastICA algorithm is applied. The inventors found that such a change can be resolved by applying clustering. In experiments performed by the inventors k-means clustering (k=8) was applied to group similar IC sources across repetitions and voluntary expressions. This utilized a cosine distance metric to compare each column in Wᵢ to that of Wⱼ, where i and j are different repetitions or expression segments. Explicitly, d_{pq}=1−COS(θ_{pq}), where d_{pq} and θ_{pq} are the distance and angle between column p in Wᵢ and column q in Wⱼ (treated as vectors), respectively. In addition to clustering similar sources to a single group, the clustering algorithm calculated the centroid's cluster for each cluster separately. Experimental results relating to these embodiments are provided in the Examples section that follows (see FIGS. 9A-D).

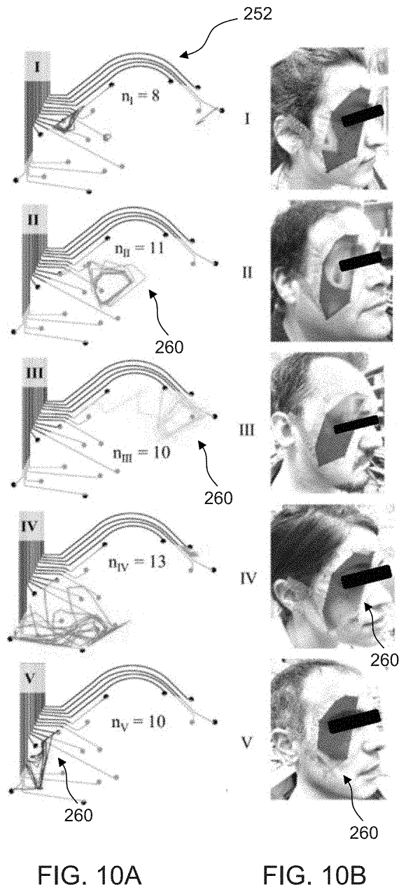

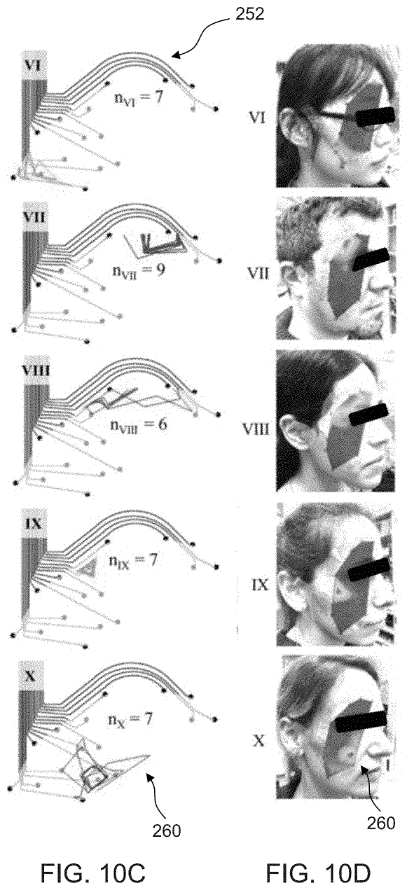

FIGS. 10A and 10C the Examples section that follows show 10 distinct groups of derived clusters for 13 subjects, in relation to a 16 electrode array. FIGS. 10B and 10D the Examples section that follows show respective IC maps corresponding to the 10 clusters, overlaid on lateral images of subjects' faces. Each is a typical IC corresponding to a consistent activation source.

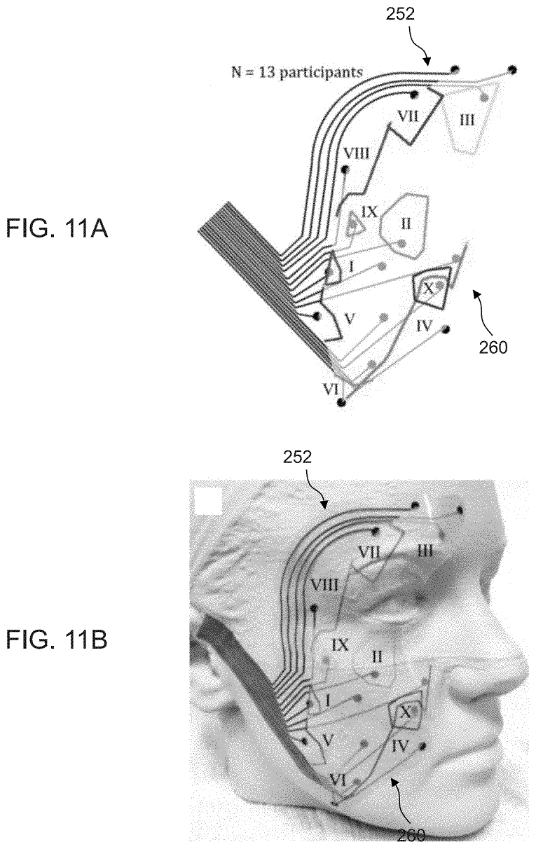

The centroid center (over subjects) can be calculated for each group by averaging all contours in that group. This is illustrated in FIGS. 11A and 11B the Examples section that follows, where FIG. 11A shows the FBBs on a hemi-facial 16 electrode array layout, and FIG. 11B shows the FBBs on a 3D model of a head.

In some embodiments, the classification algorithm relies on a k-nearest neighbor algorithm to derive the relevant FBBs for each spontaneous IC map. The classification algorithm can utilize a cosine distance metric (as detailed for the clustering algorithm above). Preferably the closest neighboring FBBs, whose distance values from the centroid center were less than a predetermined distance threshold are classified to construct a spontaneous IC source.

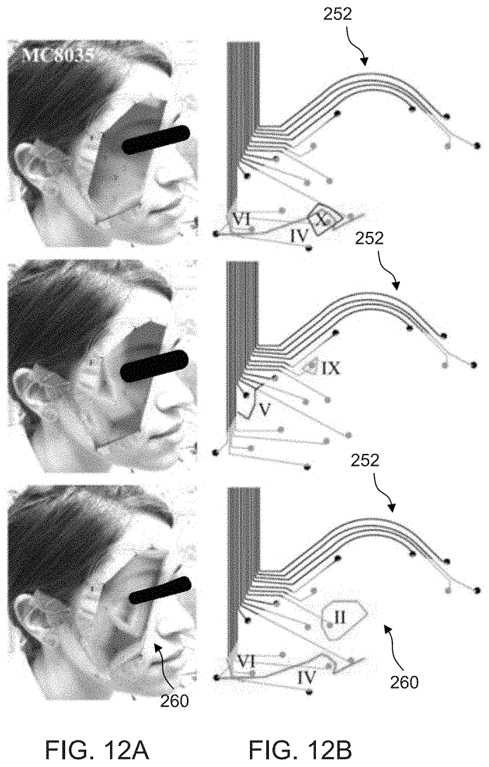

In experiments performed by the inventors, the predetermined distance threshold was set to be 0.35. Results of these experiments are shown in FIGS. 12A and 12B the Examples section that follows, where FIG. 12A shows the spontaneous IC maps overlaid on lateral images of a face, and FIG. 12B shows the spontaneous IC maps in relation to a 16 electrode array.

Each FBB can be either activated or not in a single facial expression (e.g., smile). In some embodiments, an FBB score is calculated as the number of activation occurrences divided by the number of single facial expressions in that category for each participant separately. Thus this score varies between 0 (not activated in any single facial expressions in a category) and 1 (activated in all single facial expressions in that category).

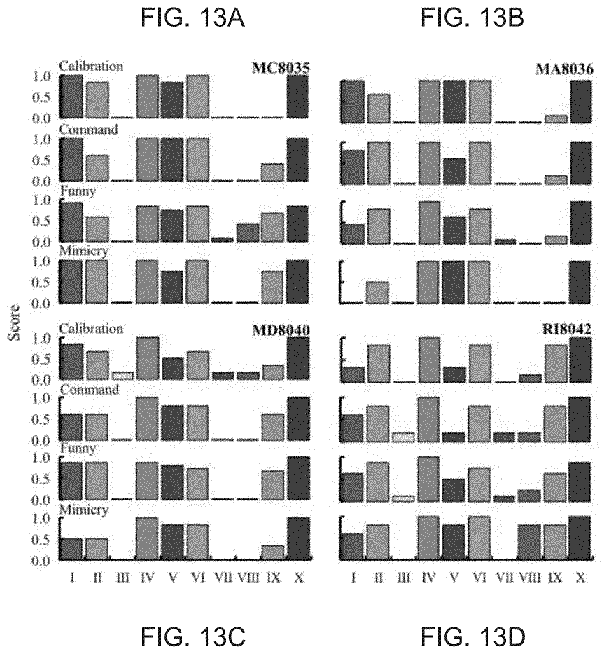

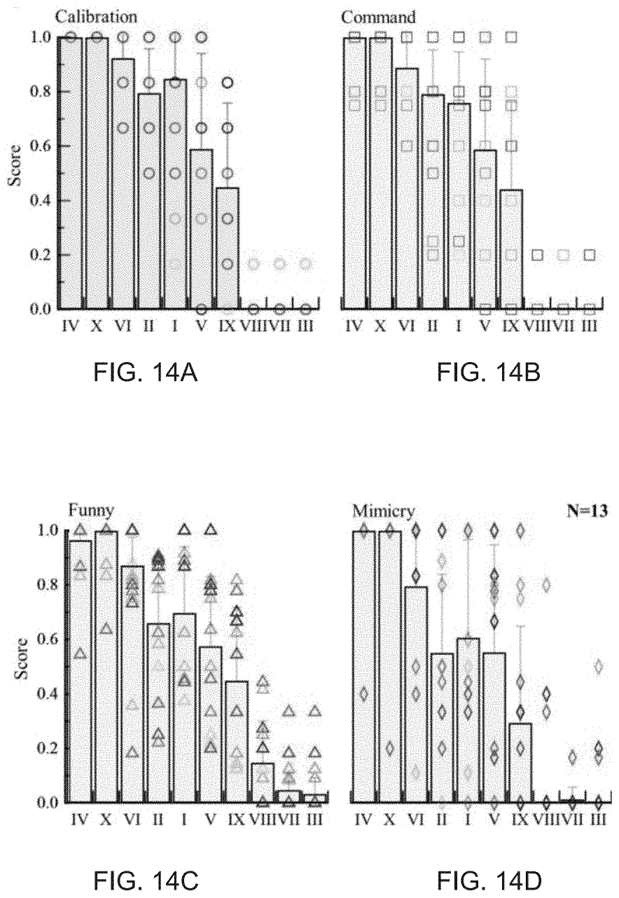

FIGS. 13A-D the Examples section that follows show FBB scores obtained for four subjects, for each of ten identified clusters I-X. This score calculation can vary to account for partial activation within the muscle with minor modifications. For a test example, a summary of all normalized FBBs in the distribution per smile category is depicted in FIGS. 14A-D. Removal of outliers from the average and standard deviation calculations typically occurs if ∈[0,Q₃+1.5·(Q₃−Q₁)], such that Q₁ is the 25% quartile and Q₃ is the 75% quartile. The range for removal of outliers can be Q₁ is in the range 10% to 35% and Q₃ is in the range 90% to 65%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

In a test of the electrode array, the left (FIG. 3) and right (FIG. 4) hemi-facial electrode arrays were connected to an amplifier unit (in the example shown, a RHD2000 amplifier board, Intan Technologies LLC) using a custom-made printed circuit board (PCB) and a zero-insertion-force (ZIF) connector. Other amplifiers, circuit boards and connectors can be used in other embodiments of the system. Preferably, the amplifier is a unipolar amplifier with high input resistance.

In the test, the array was adhered to the right side of the faces of 13 volunteer subjects (age: 31.77±7.11 years; 9 females,) after a mild skin cleaning and exfoliation. A commercial ground plate electrode (Natus Medical Incorporated; 019-409100) was positioned at the back of the neck. The array application took ˜2 min and the recorded signals stabilized after ˜2 min in all 16 channels for all participants.

In some embodiments, no separate ground electrode is needed, as the ground electrode is part of the sensing device applied to the skin. The sensing device can comprise a sensing electrode that be bipolar, a non-sensing ground electrode and any combination thereof.

In some embodiments, a ground contact is placed in other positions, for example, as an additional electrode on the face, ear or other body part. In some embodiments, the electrodes are bipolar and no ground electrode is used. The ground electrode, if used, need not be the electrode used in the test. Any commercial or proprietary electrode can be used as the ground electrode.

In this test, the software code was implemented using LabVIEW 2012 or 2017 and MATLAB R2015a. In other embodiments, any commercial or proprietary analysis software can be used.

In a test of the system, analysis of facial expressions using the electrode array of FIGS. 3 and 4 and the associated software had a measurement part that lasted about an hour and comprised two steps; a calibration step of voluntary expressions and a spontaneous step of different smile types.

In a test of the system, in an introductory step, the user is shown a sample of photographs and text of four expressions: a voluntary-smile (FIG. 7A), close-the-eyes-forcefully (FIG. 7B), contract-the-eyebrows (FIG. 7C) and press-the-lips-together (FIG. 7D), to ensure that the user understands the facial expressions to be exhibited in the calibration and spontaneous steps. Each expression is presented for 3 s followed by a 3 s gap, during which a neutral expression is showed. Each facial expression is presented 3 times consecutively before the spontaneous step and 3 times after (total of six repetitions).

In a test of the embodiment, thirty-three videos were presented during the spontaneous step (duration range: 5 to 39 s) separated by 7 s of a blank slide (total time was 15 min 23 s). The number of videos can range from 1 to 100, and the length of each video range from 5 s to 10 min. More than one expression can be elicited during a single video.

Users were instructed to watch the videos and react spontaneously. In addition, users were instructed to perform facial expressions as a response to written command tasks shown on the screen (e.g. “Smile as if you saw your best friend”). This step involved three types of scene: (1) funny episodes (i.e. funny) (N_{f}=16); (2) individuals smiling to the camera (i.e. mimicry) (Nₘ=12); and (3) written instructions to smile (i.e. command) (N_{c}=5). N_{f}, Nₘ, and N_{c} can each be in the range from 5 to 30, with the number of videos for each expression dependent on the total number of videos used. Preferably, the number of is approximately the same for the different types of facial expression. FIGS. 8A-8D illustrate typical responses, overlaid on a neutral image of the face, showing muscle activation patterns for the four expressions, a voluntary-smile (FIG. 8A), close-the-eyes-forcefully (FIG. 8B), contract-the-eyebrows (FIG. 8C) and press-the-lips-together (FIG. 8D), where blue indicates areas of low response, shading through to red, which indicates significant muscle activation. The voluntary-smile pattern (FIG. 8A) and the press-the-lips-together pattern (FIG. 8D) are the most similar. For both the voluntary-smile pattern (FIG. 8A) and the press-the-lips-together pattern (FIG. 8D), the strongest muscle activation is in the lower cheek region, near the lips. These are clearly different from the close-the-eyes-forcefully pattern (FIG. 8B) and the contract-the-eyebrows (FIG. 8C), the former having the strongest muscle activation in the forehead near the nose and the latter having the strongest muscle activation in the upper part of the cheeks, near the outer edge of the eye.

In the test, sEMG and the videos were recorded simultaneously for later evaluation. In use, only sEMG is necessarily recorded; video or other visual recordings can also be made. For the visual recordings during the test, the users were laterally photographed; these photographs included images taken of neutral facial expression for later analysis (used for the blind source separation algorithm step).

In this test, data analysis was performed using MATLAB R2017ab and R2018b. sEMG data were recorded with a sampling rate of 3000 samples/s; the sampling rate can be in a range from 2000 samples/s to 5000 samples/s. Data were filtered using a 50 Hz comb filter and a bandpass 4 order Butterworth filter in the frequency range of 20-500 Hz. Other filters and filtering frequencies can be used in other embodiments. Any commercial or proprietary data analysis software with the appropriate capabilities can be used Examples include, but are not limited to, HubSpot Analytics, Qlik, Alteryx, NumPy, Stata, PARIS, Base SAS, SAS Enterprise Miner, HPE Vertica and SAS/STAT.

sEMG segments were cut as follows: Calibration step: 3 s before voluntary task instructions commencement and 3 s after termination. Spontaneous step: 1 s before video commencement and 6 s after video termination. The adapted fastICA was applied for the 16 single-channel sEMG data for each voluntary facial expression (six repetitions) separately and for each video segment separately. Different pre-step and post-step cut times different numbers of repetitions can be used in other embodiments. Pre-step and post-step cut times can be in the range from 0 s to 20 s and the number of repetitions can be in the range from none (once for at least one repetition) to 20 repetitions.

The printed hemifacial 16 electrode array disclosed herein has a high inter-electrode density to cover both upper and lower lateral parts of the face. The same electrode array layout was used for all participants (N=13). In the calibration step, each participant sat in a relaxed upright position and was instructed to perform four voluntary expressions (described by photographs and text on a computer screen). Typical sEMG results are shown in FIG. 6; the expressions, a voluntary-smile, contract-the-eyebrows, close-the-eyes-forcefully and press-the-lips-together shown in FIG. 7A-D. These specific expressions where chosen as they are known to activate a large number of muscles.

FIG. 6 shows single channel sEMG data recorded from electrodes 1, 2, 6, 8, 9, 12, 13 and 15 from a single participant (participant MC8035). Both voluntary-smile and press-the-lips-together are seen as an elevation in amplitude in electrodes 1, 2, 6, 8, 9 and 15. Contract-the-eyebrows was recorded primarily at electrodes 12 and 13 and close-the-eyes-forcefully was apparent mostly at electrodes 8, 9, 12 and 13.

While the data in FIG. 6 reveal some discrimination between different expressions, strong cross-talk prevents the identification of specific muscle activation; a fastICA algorithm can be used to better identify specific muscle activation. Source localization can also identify anatomical differences between participants. The electrode location and the inverse un-mixing matrix, W, can be used to reveal the IC maps for each voluntary facial expression in each repetition separately (see hereinbelow). FIG. 8A-D depicts four derived IC sources (primary) of one participant in the calibration step. A primary IC can be defined as one that recurs in all repetitions within a voluntary expression; e.g., contract-the-eyebrows activated an area above and around the right eyebrow in all six repetitions (FIG. 8B; red color indicates the highest muscle activation and blue the lowest).

FIG. 9A-D shows IC sources computed and clustered from the calibration step (participant MC8035). Seven out of 8 clusters are presented. Clusters are numbered by roman numerals and are color-coded (I-red, II-orange, III-yellow, IV-light green, VI-light blue, VII-dark blue, VIII-purple). FIG. 9A shows normalized un-mixing matrix weights at each electrode (mi is the number of ICs within a cluster). Black dashed lines represent the normalized centroids of each cluster. FIG. 9B shows distance from cluster centroids, FIG. 9C schematically illustrates a spatial representation of the IC sources as a contour form on the 16 electrode array layout and FIG. 9D schematically illustrates contours of cluster centroids on the 16 electrode array layout.

In addition to the primary sources, fastICA also revealed additional secondary sources for each expression (identified by a weight in each electrode, cells of columns in the un-mixing matrix, W). A clustering algorithm groups similar IC sources across repetitions and voluntary expressions. FIG. 9A-C demonstrates 113 IC sources (out of 124) clustered into 7 (out of 8) spatially clean and separable clusters (I, II, III, IV, VI, VII, VIII) for a single participant (11 IC sources were clustered into a group that overlapped other clusters): 24 ICs were derived from the contract-the-eyebrows task; 21 from press-the-lips-together; 33 from close-the-eyes-forcefully; and 46 from the voluntary-smile task. The normalized un-mixing matrix weights at each electrode for all clusters are depicted in FIG. 9A. Each color represents a different cluster (I-red, II-orange, III-yellow, IV-light green, VI-light blue, VII-dark blue, VIII-purple). Black dashed lines depict the clusters' centroids. The sources' distance from their cluster's centroid is shown in FIG. 9B. FIG. 9C shows the 113 clustered IC sources (color-coded) plotted one on top of the other in contour form on the 16 electrode array layout. Each IC contour was spatially defined by the maximal muscle activation location at the IC map minus 1.5 standard deviations from that maximum. The cluster centroid contours are color- and number-coded in FIG. 9D. The contours were derived from all of the calibration tasks (voluntary-smile, close-the-eyes-forcefully, contract-the-eyebrows and press-the-lips-together) and are not expression specific. A second important point is that few sources appear to be much less consistent than others since some muscle contractions appear in pronounced movements, as discussed hereinbelow.

In a test of the system, the mathematical scheme described above was repeated for each of the 13 participants in the test and the derived clusters were grouped together. This procedure resulted in 10 distinct clusters (numbered by roman numerals I-X in FIGS. 10A and 10C). FIGS. 10A and 10C summarize the centroid cluster contours for all individuals (color-coded), e.g., cluster I depicts 8 (out of 13 participants) that had source I activated during the calibration step. Each contour in FIGS. 10A and 10C is a centroid of all IC contours, derived for each individual separately. It is important to note that most centroid clusters are extremely consistent across participants, although a few seem to be more scattered. For clarity, FIGS. 10B and 10D show 10 examples of corresponding maps for different individuals. Summarizing these results, FIGS. 11A and 11B show the centroid centers on top of the 16 electrode array layout and on a 3D human model, respectively. Here, each contour is the mean of all contours of the same cluster, e.g., the red contour is the center of all eight centroid contours of group I, as shown in FIGS. 10A and 10C.

In this test, 10 consistent FBBs were found (FIGS. 13A-D and Table 1). Some FBBs formed a dichotomic pattern; they were activated in some expressions in some participants (>5 individuals) and were absent (<5) in others. Close-the-eyes-forcefully and contract-the-eyebrows consistently activated building blocks III, VII and VIII (upper part of the face). Voluntary-smile and press-the-lips-together activated building blocks IV and VI (lower part of the face). Building blocks II and IX were activated in voluntary-smile and close-the-eyes-forcefully (eye-region). Finally, associating the FBBs with specific muscles (and their sections) was performed based on their location in space and their functionality. For instance, FBBs I and X were associated with the Zygomaticus major and Zygomaticus minor muscles, respectively. FBBs II and IX were linked to the lower preorbital section and the lower-lateral section of the Orbicularis oculi. FBB IV was activated in all 13 participants during smiling. Closer observation of FBB IV revealed two activation patterns: Zygomaticus major activation alone (6 out of 13 subjects) or simultaneous activation of two regions: Zygomaticus major (lower face) and Glabellar muscles (upper face) (7 out of 13 subjects) (FIGS. 10B and 10D). Mean calculation of the centroid centers for FBB IV resulted in a single activation source (FIGS. 11A-B and Table 1). Further discussion regarding FBB IV is found hereinbelow.

### Table 1

Number of participants (out of 13) that activated a specific (well separated) FBB during the calibration step. Facial muscles were identified for each FBB. The 10 FBBs are depicted in roman numerals (I-X)

| | | | Close | | |
|---|---|---|---|---|---|
| | Volun- | Press | the eyes | Contract | |
| Building | tary | the lips | force- | the eye- | |
| blocks | smile | together | fully | brows | Muscle |
| III | 0 | 0 | 8 | 10 | Corrugator supercilii |
| VII | 0 | 1 | 8 | 8 | preorbital Orbicularis |
| | | | | | oculi (upper section) |
| VIII | 1 | 0 | 5 | 5 | preorbital Orbicularis |
| | | | | | oculi (upper-lateral |
| | | | | | section) |
| IV | 13 | 13 | 4 | 2 | Zygomaticus major |
| VI | 6 | 6 | 2 | 4 | Risorius |
| II | 11 | 3 | 10 | 2 | preorbital Orbicularis |
| | | | | | oculi (lower section) |
| IX | 7 | 1 | 5 | 3 | preorbital Orbicularis |
| | | | | | oculi (lower-lateral |
| | | | | | section) |
| V | 7 | 5 | 9 | 6 | Masseter/Platysma |
| | | | | | branches/Risorius |
| I | 7 | 3 | 8 | 7 | Zygomaticus major |
| X | 7 | 5 | 6 | 0 | Zygomaticus minor |

With an objective process to derive individual (FIG. 9A-D) and common (FIGS. 10A-11B) FBB mappings, these maps can be used to identify FBB activation in spontaneous smiling. In a test, the 13 participants from the initial test were instructed to watch a series of 33 short videos. The experiment involved three video types: (1) funny episodes (i.e. funny); (2) individuals smiling to the camera (i.e. mimicry); and (3) written instructions to smile (i.e. command). Owing to the large number of muscles activated during smiling, IC sources identified from the spontaneous data were seldom the clean FBBs observed in the calibration stage. Rather, they were often a combination of several FBBs (FIGS. 12A-B, where FIG. 12A shows the IC maps overlaid on a user's face and FIG. 12B shows the IC maps in relation to a 16 electrode array). The classification algorithm to derive the relevant FBBs for each IC map as disclosed hereinabove was applied to the smiles. Each video segment consisted of several ICs, or, in other words, each video segment was geometrically spanned by a number of FBBs. These FBBs were saved for each video segment separately. FIGS. 13A-D show normalized histograms of FBBs for four individuals (MC8035, MA8036, MD8040 and RI8042) in the three spontaneous step categories (command, funny and mimicry) and the calibration step. Each individual appears to have a consistent, yet distinct, signature, which is independent of the smiling category, with some FBBs showing a score of 1 (FBBs IV and X) and others a score which is subject specific (FBBs IX and V for example).

A summary of all normalized FBBs distributions per category is depicted in FIGS. 14A-D for all 13 individuals. The results show a typical (average) FBB activation pattern which is consistent between smile categories. In this test, no difference was found between spontaneous smiles, such as in funny, to those in command or calibration. Specifically, FBBs IV and X had a score of almost 1 in all categories, whereas FBBs VIII, VII and III had a score of almost 0 (with the exception of the funny category). The other FBBs were more variable, with greater differences between individuals. These results echo previous findings suggesting that smiles that involve the Zygomaticus major (FBBs IV or I) and the Orbicularis oculi (e.g. FBBs II or IX) together (such as a Duchenne smile) could be produced both spontaneously and voluntarily. Moreover, smiles that activate the Zygomaticus major alone (such as a non-Duchenne smile) could be also produced deliberately and spontaneously [Krumhuber E G and Manstead A S R, Can Duchenne smiles be feigned? New evidence on felt and false smiles. Emotion 9 807-20, 2009; Maringer M, Krumhuber E G, Fischer A H and Niedenthal P M, Beyond smile dynamics: Mimicry and beliefs in judgments of smiles. Emotion 11 181-7, 2011; Rychlowska M, Cañadas E, Wood A, Krumhuber E G, Fischer A and Niedenthal P M, Blocking Mimicry Makes True and False Smiles Look the Same. ed M Iacoboni PLoS One 9 e90876, 2014]. Explicitly, FBB II, the lower preorbital Orbicularis oculi has an average score of 0.66 in funny smiles and scores of 0.79 and 0.8 in deliberate smiles (calibration step and command smiles, respectively). These results also highlight the individual nature of each person's smiling pattern and the ability of our technique to reveal it at an unprecedented resolution in a non-invasive manner.

For facial activation, the system of the present invention can identify and cluster sEMG IC sources from voluntary facial activation and can consistently classify them while allowing participants to freely move their heads and faces.

For facial activation, the system can robustly identify 10 separate activation FBBs and associate them to six facial muscles and their sections. The smile signature appears to be similar among smile types, comprising the activation of the Zygomaticus major with or without the Orbicularis oculi.

In another test, a high-resolution electrode array combined with statistical analysis enabled robust classification of complex forearm muscle activity at varying contraction levels.

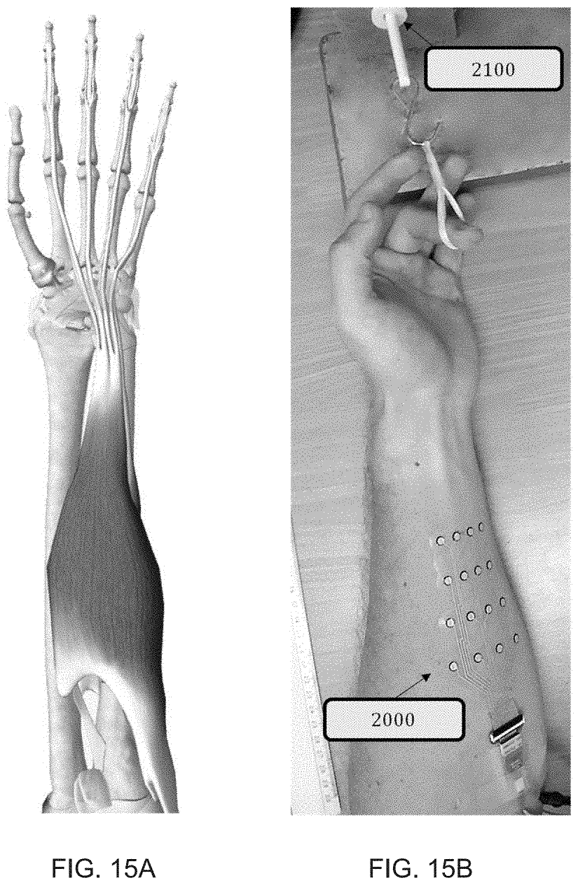

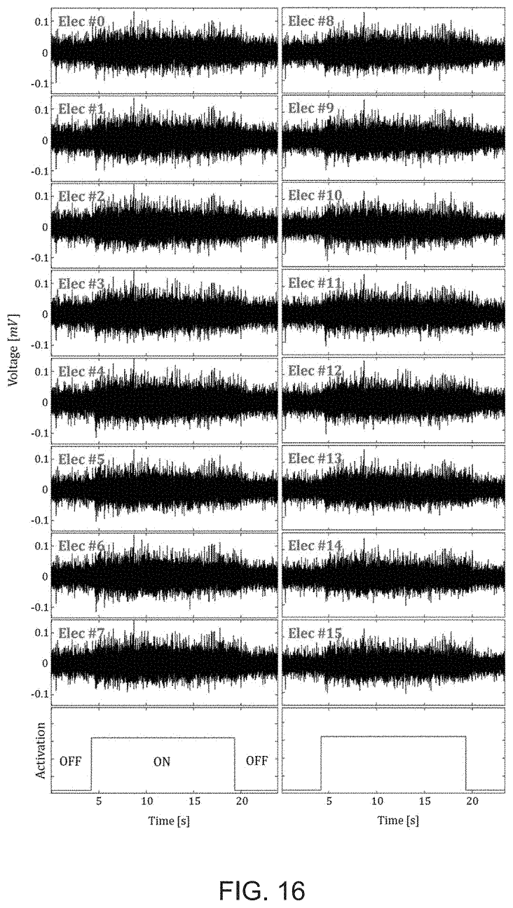

The high-resolution electrode array 2000 was used to record sEMG from the forearm (FIGS. 15A-B).

In order to improve the accuracy of the weighting of the electrode responses, the muscle contraction level during finger flexion was measured using a force gauge 2100, for middle finger flexion against a spring.

As shown in FIGS. 15A-B, in this test, an array of 15 electrodes was placed above the flexor digitorum muscle and connected to an Intan RHD2000 amplifier. FIG. 15A shows the flexor digitorum muscle and FIG. 15B shows the device on a forearm. The electrodes (2000) are adhered to the forearm and muscle contraction levels during finger flexion were measured using a force gauge (2100). In other embodiments, any commercial or proprietary amplifier with an appropriate amplification and an appropriate power range can be used.

FIG. 16 shows typical results as recorded by the electrodes in the electrode array.

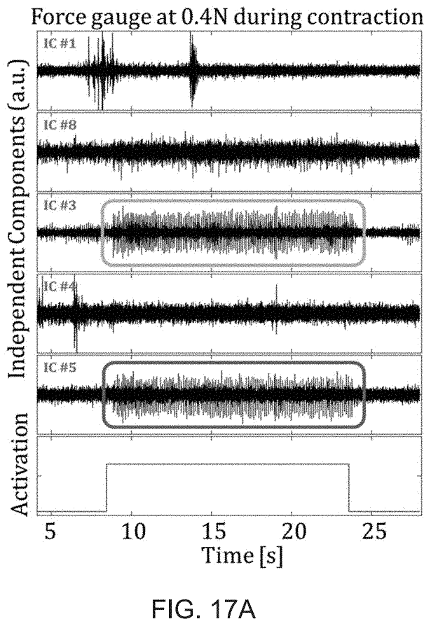

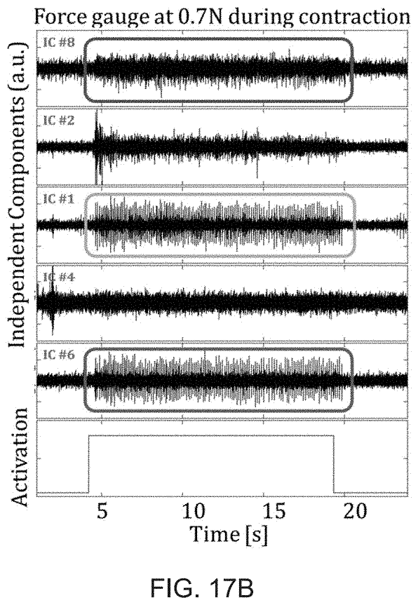

FIG. 17A-B shows independent components of the contracting flexor digitorum muscle at different levels of exerted force, with FIG. 17A showing results for a force of 0.4N and FIG. 17B showing results for a force of 0.7N. The colored regions relate to specific activation sources. The other ICs contain noise.

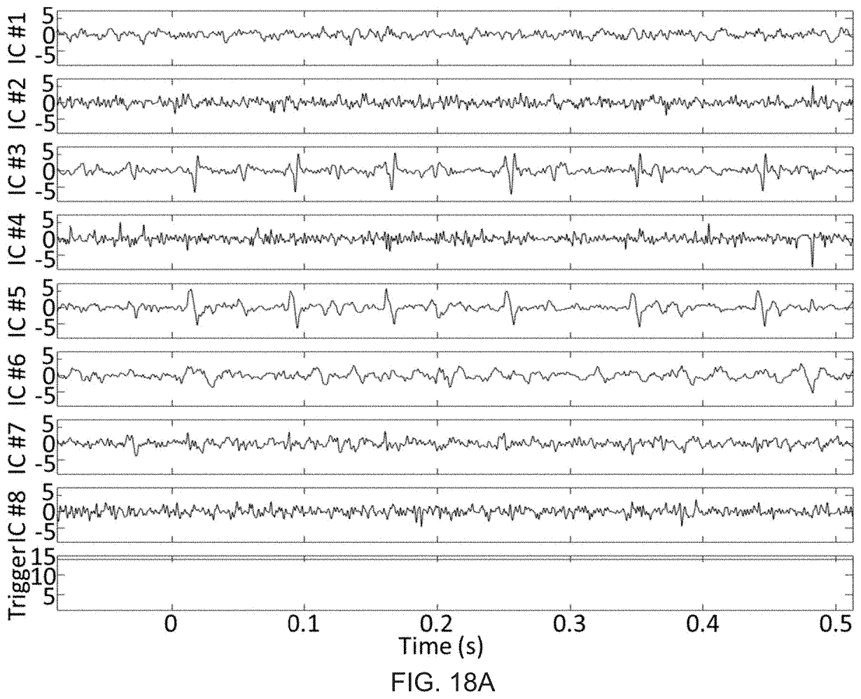

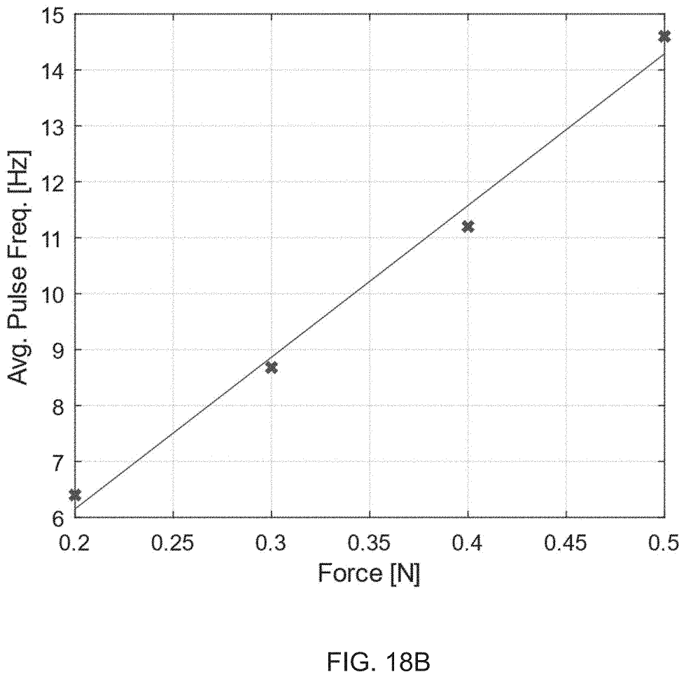

Muscle unit action potential (MUAP) activity can be detected after applying ICA. FIG. 18A shows two components with MUAP pulse trains while performing flexion under 0.5N force. FIG. 18B and Table 2 show the linear relationship between pulse frequency (MUAP Rate (MR)) and force [Kallenberg L A C and Hermens H J, Behaviour of motor unit action potential rate, estimated from surface EMG, as a measure of muscle activation level. J Neuroengineering Rehabil. 3, 2006]. To separate physiologically interesting components from the noisy data a binary classifier was built, as described hereinabove, which qualifies sEMG data using the tempo-spectral parameters of each independent component.

TABLE 2

| Force (N) | Frequency (Hz) |
|---|---|
| 0.2 | 6.40 |
| 0.3 | 8.68 |
| 0.4 | 11.2 |
| 0.5 | 14.6 |

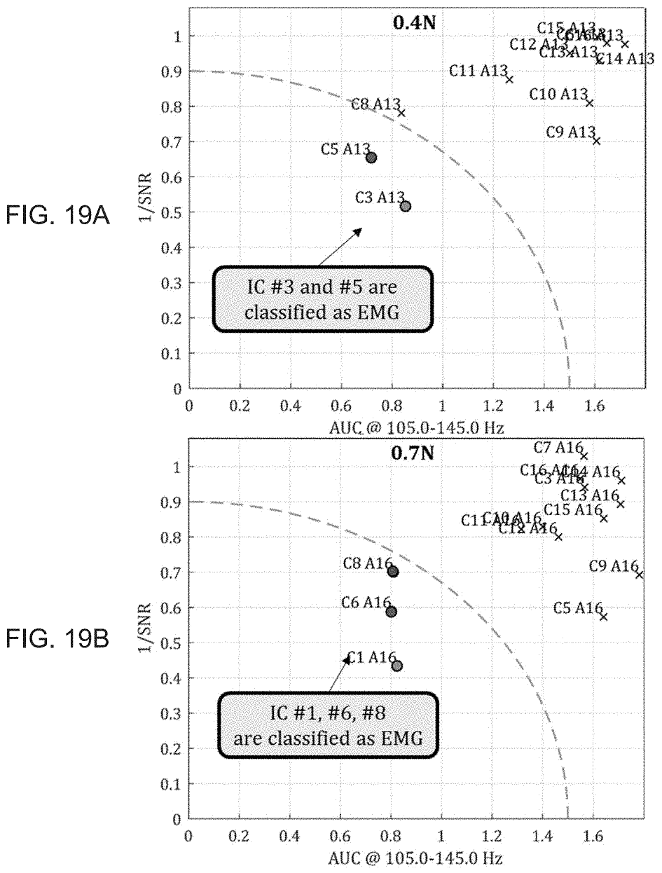

FIGS. 19A-B show separating physiologically interesting components from the noisy data by building a binary classifier which qualifies sEMG data using tempo-spectral parameters of each IC. The dashed curve is the classifier. SNR is Signal-to-Noise ratio. AUC is Area Under the Curve of the spectrum in a range of frequencies; in this example, the range was 105 Hz to 145 Hz.

The classified ICs (muscle activity sources) were sorted using a similarity metric. They were mapped onto the electrode positions according to their fastICA mixing matrices.

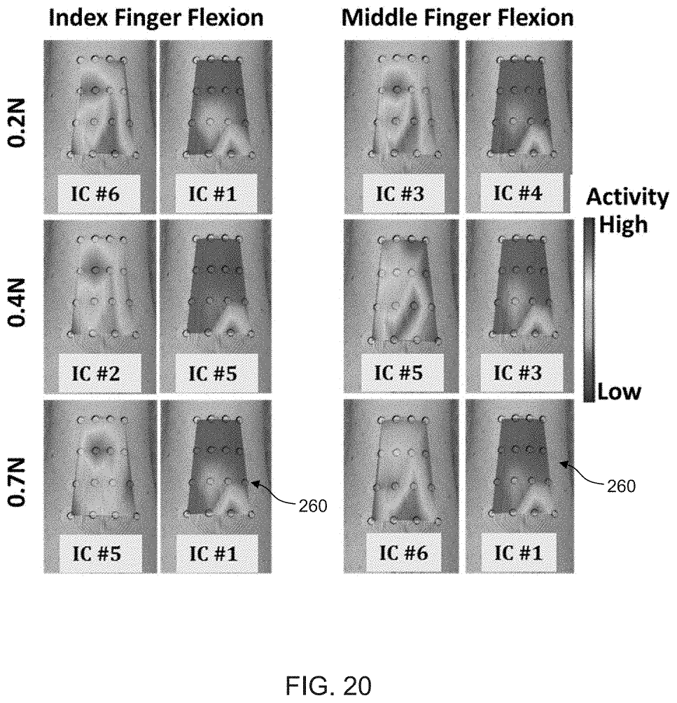

FIG. 20 shows activation of the flexor muscles for finger flexion for forces of 0.2N, 0.4N and 0.7N.

Given a set of tasks which utilize flexor muscles in the forearm (flexor carpi radialis, flexor pollicis longus, flexor digitorum superficialis, flexor digitorum profundus, flexor carpi ulnaris, pronator teres and palmaris longus) it is possible to show the spatial distribution of extracted independent components over the area covered by the electrodes.

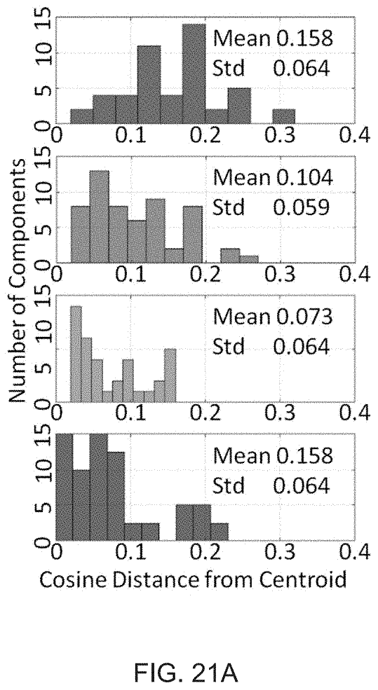

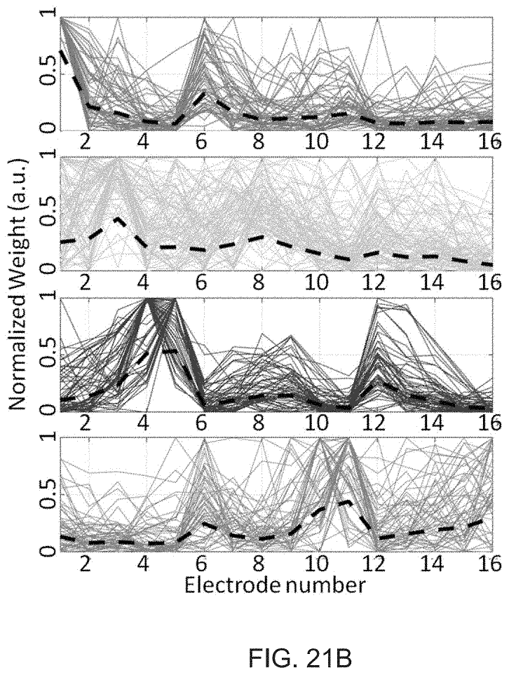

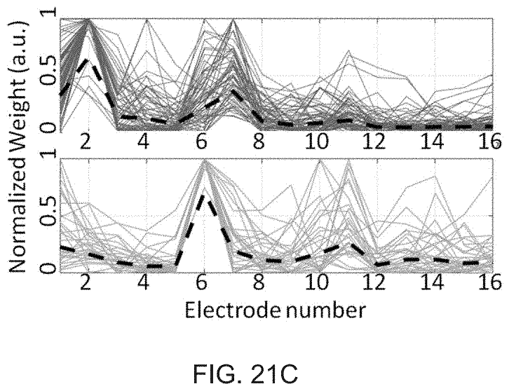

FIGS. 21A-C shows independent components extracted from different flexion tasks as normalized weights distributed over 16 electrodes and sorted into 10 clusters using cosine distance k-means.

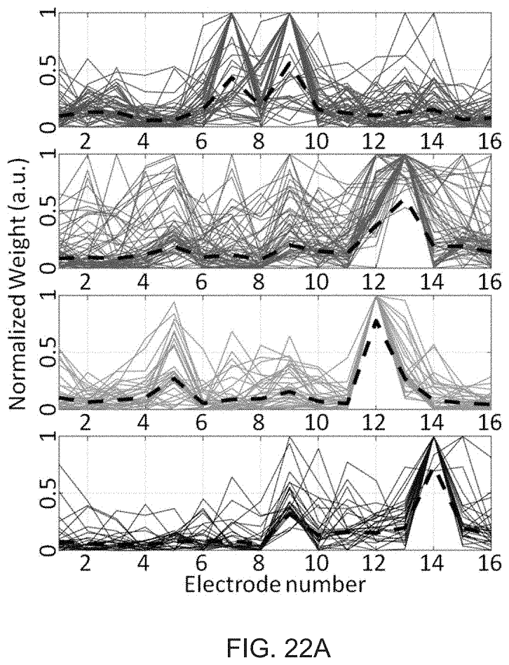

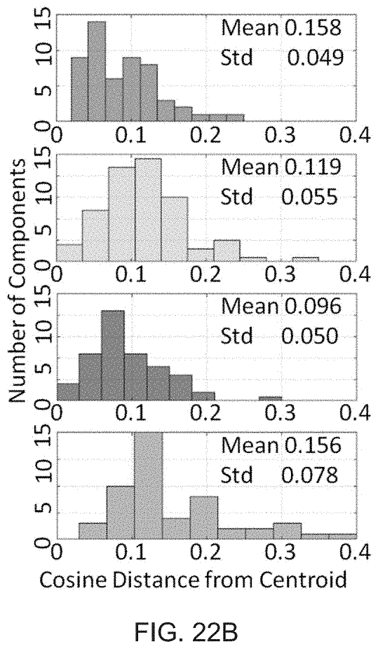

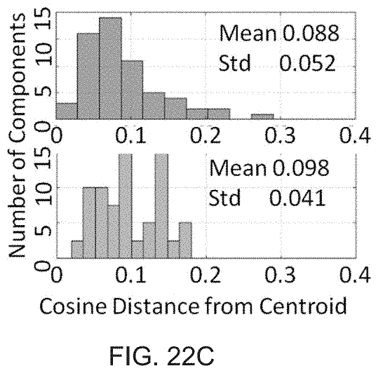

FIGS. 22A-C shows the cosine similarity between the components in FIG. 20.

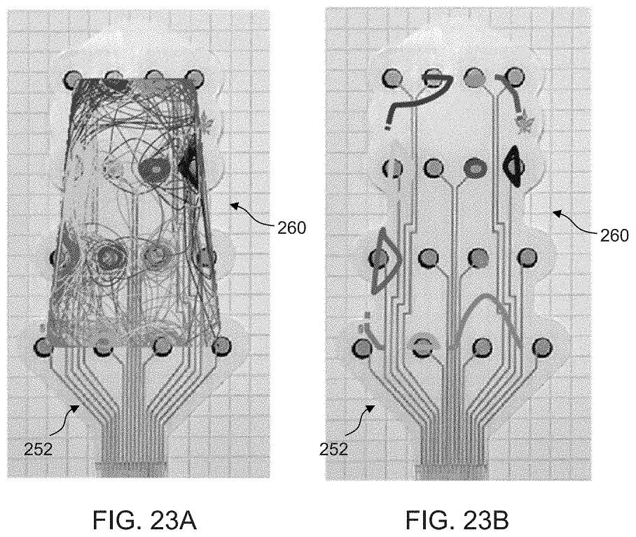

FIG. 23A shows ten independent components clusters projected onto the area covered by the electrodes using representative contour lines.

FIG. 23B shows the centroids of the independent component clusters from FIG. 23A.

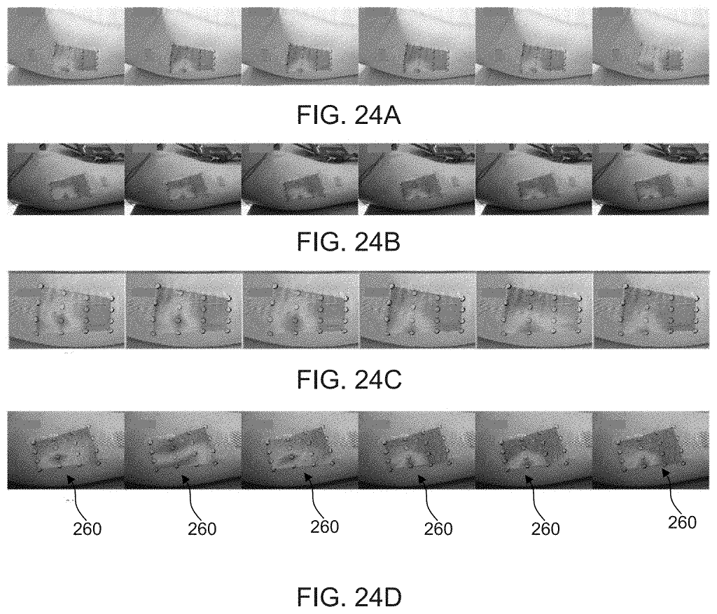

FIGS. 24A-D each show six repetitions of an activity, with FIGS. 24A and 24C showing activity areas with the electrodes in a first orientation and FIGS. 24B and 24D showing activity areas with the electrodes in a second orientation. FIGS. 24A-B show the activity of the Palmaris longus, and FIGS. 24C-D show the activity of the flexor digitorum superficialis using ring finger flexion.

As shown in FIGS. 24A-D, the extracted IC's are invariant to electrode array orientation and the activity areas remain similar, as shown in FIGS. 24A-B, and in FIGS. 24C-D.

The system of the present embodiments can be used hemi-facially or bilaterally, e.g., on only one side of the face or on both sides; similarly, electrodes can be attached to one limb or both, and to one side or both sides of a portion of a torso.

The system of the present embodiments can be used in sports for determining muscle fatigue, for performance training, for rehabilitation, for determining muscle pain and any combination thereof. If a parameter, for non-limiting example, pain, is outside at least one predetermined limit, a warning can be provided. The warning can be provided visually, audibly or tactilely, and can be provided to a user, to another person, stored in a database, and any combination thereof.

The system of the present invention can be used in relation to plastic surgery, for improvement of facial symmetry, during rehabilitation physiotherapy and any combination thereof. Again, a warning can be provided if a parameter is outside at least one predetermined limit. The warning can be provided visually, audibly or tactilely, and can be provided to a user, to another person, stored in a database, and any combination thereof.

The system of the present invention can be used to determine drug toxicity, as a biomarker, to identify bruxism and any combination thereof. A facial change can be a marker for a disease, a stroke, paralysis, brain damage, a brain tumor and any combination thereof.

The system of the present invention can be used in neurorehabilitation and for characterization of walking, assessment of post-stroke and post-spinal cord injury motor recovery, spasticity assessment, biofeedback and “serious games”, study of muscle synergies, control of prosthesis, exoskeletons and robots, body-machine interfaces, non-invasive extraction of neural control strategies, myoelectric manifestations of muscle fatigue, cramps, and any combination thereof.

The system of the present invention can be used before, during or after a surgical intervention.

The electrode set of the present embodiments can be a sticker, a temporary tattoo, or any other method of soft adhering electrodes that can be positioned at a specific portion of a body. Preferably, the electrode set is temporarily adhered to the body. In some embodiments, the electrode set can remain functional on the body for a period of up to one week.

In some embodiments, the detected muscle activation can be used to control a robot or a smart appliance.

The connection between an electrode set and the system comprising the analysis software can be wired or wireless. Preferably, the connection is wireless.

Other embodiments of the system can provide home-based monitoring of patients with ailments affecting the respiratory and cardiovascular functions, such as, but not limited to, corona virus disease 2019 (COVID-19), pneumonia, and influenza.

In such embodiments, the system comprises a telemetric device to monitor respiratory and cardiac measures of the patients at early stages of the disease, such as while quarantined at home or at a dedicated quarantine center. The device is designed to provide an alert for a transition from a mild to a severe manifestation of the disease, at the point where at least one of the patient's respiratory and cardiovascular function begins to deteriorate. The technology can provide real-time data to assist a clinician in the decision on whether or not to provide more aggressive treatment, such as, for non-limiting examples, transferring a remote patient to a hospital or transferring a patient in a hospital to an intensive-care ward.

An example of an application of such embodiments is provided by the COVID-19, a pandemic that creates a tremendous load on healthcare systems worldwide. The clinical spectrum of the disease varies from asymptomatic to conditions that involve respiratory failure, such as in severe acute respiratory distress syndrome (ARDS), which necessitates mechanical ventilation and support in an intensive care unit (ICU). ARDS is characterized by the development of acute shortness of breath (dyspnea) and deficiency of oxygen in the blood (hypoxemia) within hours to days of an inciting event. Physical findings of ARDS often are nonspecific and include abnormal rapid breathing (tachypnea) and heartrate (tachycardia). Fever, which may increase the heart rate, is associated with severe dyspnea, but it can be moderate or even absent. Moreover, cardiovascular implications are also reported in the disease especially in, but not limited to, patients with preexisting cardiovascular disease. COVID-19 has been associated with multiple direct and indirect cardiovascular complications including myocardial injury, myocarditis, arrhythmias and venous thromboembolism (Interim Clinical Guidance for Management of Patients with Confirmed Coronavirus Disease (COVID-19). Centers for Disease Control and Prevention www(dot)cdc(dot)gov/coronavirus/2019-ncov/hcp/clinical-guidance-management-patients(dot)html (2020)). The Chinese Center for Disease Control and Prevention (CDC) report divided the clinical manifestations of COVID-19 by severity: mild, non- and mild pneumonia, accounted for 81% of cases; severe, characterized by development of ARDS with respiratory frequency increase above 30 cycles per minute, occurred in 14% of cases; critical, characterized by respiratory failure, septic shock, and/or multiple organ dysfunction, occurred in 5% of cases. Other reports suggest that over half of the patients with confirmed COVID-19 and pneumonia develop dyspnea 5-13 days after illness onset, and that ARDS develops in 17-29% of hospitalized patients (Kangelaris, K. N. et al. Timing of intubation and clinical outcomes in adults with acute respiratory distress syndrome. Crit. Care Med. 44, 120-129 (2016)). Though severe coronavirus cases have been reported among younger and middle-aged adults, older adults, the elderly and those with chronic health conditions seem to be most at risk for the sudden decline that leads to ARDS. This seems to be the crucial phase of the disease. From this point onwards there may be a rapid deterioration of respiratory functions (Cascella, M., Rajnik, M., Dulebohn, S. C. & Di Napoli, R. Features, Evaluation and Treatment Coronavirus (COVID-19). (StatPearls Publishing LLC, 2020)), and timing for intubation and mechanical ventilation critical, since late intubation is associated with increased mortality (Cabral, E. E. A. et al. Surface electromyography (sEMG) of extradiaphragm respiratory muscles in healthy subjects: A systematic review. J. Electromyogr. Kinesiol. 42, 123-135 (2018)). Once diagnosed, patients with a mild clinical manifestation are quarantined at home or at dedicated quarantine centers. Thus, it is of extreme importance to remotely monitor their respiratory and cardiovascular functions, to detect early signs of deterioration, and to assist the clinician to decide on whether and when to transfer a patient to a hospital or other more intensive care facility.

The respiratory muscles are vital to produce adequate ventilation and gas exchange. Contraction of the respiratory muscles creates a negative pressure gradient that results in inflow of air into the lungs. The diaphragm performs the largest portion of the inspiratory process, together with several other muscles which contribute to inspiration and expiration (Gibson, G. J. et al. ATS/ERS Statement on respiratory muscle testing. Am. J. Respir. Crit. Care Med. 166, 518-624 (2002)). EMG signals can be analyzed to determine normal and abnormal function of the neuromuscular system, including the respiratory muscles (Luo, Y. M. & Moxham, J. Measurement of neural respiratory drive in patients with COPD. Respir. Physiol. Neurobiol. 146, 165-174 (2005)).

EMG monitoring of the respiratory muscles has been evaluated in a variety of clinical and experimental scenarios. Traditionally, quantifying the diaphragmatic electromyogram (EMGdi) activity has been performed by using a multipair esophageal electrode catheter that is swallowed by patients (also called transesophageal EMGdi; esEMGdi) (Wu, W. et al. Correlation and compatibility between surface respiratory electromyography and transesophageal diaphragmatic electromyography measurements during treadmill exercise in stable patients with COPD. Int. J. COPD 12, 3273-3280 (2017)). This technology is invasive and leads to discomfort during EMGdi detection. Furthermore, the complex operation and the discomfort experienced by patients reduces follow-up visits and increases the loss rate. On the other hand, as discussed above, sEMG decreases the pain associated with the procedure, increases patient compliance and can provide continuous monitoring. Moreover, surface inspiratory EMG activity, recorded from the diaphragm (sEMGdi), the parasternal intercostal muscle (sEMGpara), and from the sternocleidomastoid (sEMGsc) are closely related to the esEMGdi. Therefore, surface measurements of respiratory muscle activity are a reliable method of detecting breathlessness and respiratory disorders, such as are manifested in COVID-19. In the prior art, however, sEMG still required the presence of a clinician or other formal medical setup.

Moreover, as COVID-19 has implications for the cardiovascular system as well, ongoing monitoring of the heart, as in ECG can be crucial for evaluating the severity of the disease progression.

An exemplary embodiment of measuring the sEMG signal is shown in FIG. 26. In this exemplary embodiment, an 8 channel multi-electrode array was placed below the sternum. The user held his breath for 20 s, followed by 8 deep breaths and one shallow breath.

FIG. 27A shows the signal, with FIG. 27A showing the recorded differential signal of electrodes 0-7 and FIG. 27B showing the smoothed signal of the sEMGdi. The EMG signal is overlapped with the heart's ECG.

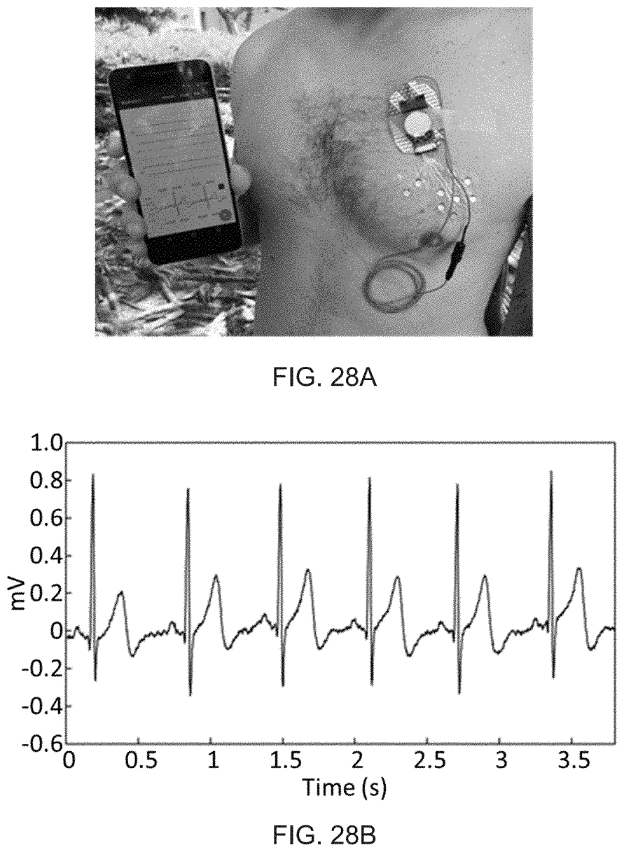

An exemplary embodiment of measuring the ECG is shown in FIGS. 28A-B. In this exemplary embodiment, an 8 channel multi-electrode electrode array was placed on the left side of the chest (FIG. 28A). FIG. 28B shows the recorded ECG signal from a single channel.

The sEMG data and the ECG data can be analyzed as disclosed above, and an alert, typically at a remote location but, in some embodiments, at a local location, can be provided at such time as at least one of the heart function data and the muscular function data show a change indicating increased severity of the illness and a need for further intervention, as discussed above.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for determining muscle activation comprising:
a set of electrode adherable to a skin of a subject; and
a processor in communication with the electrodes, and having a circuit configured for receiving locations of said electrodes and electrical signals detected by said electrodes, analyzing said signals to identify a section of an active muscle, identifying locations of at least a segment of active muscles and activation patterns of said active muscles based on said identified section, and constructing a displayable map of said locations and said activation patterns, wherein patterns corresponding to different active muscles are distinguishable on said map.

2. The system of claim 1, wherein said map overlays an image of a body portion and/or a graphical representation of said electrodes.

3. The system of claim 2, wherein said body portion is selected from a group consisting of a portion of a face, a portion of a neck, a portion of an arm, a portion of a leg, a portion of a hand, a portion of a foot, a portion of a torso, a portion of a head, and any combination thereof.

4. The system according to claim 1, wherein said analysis is carried out by a blind source separation algorithm.

5. The system of claim 4, wherein said blind source separation algorithm comprises an algorithm selected from a group consisting of independent component analysis (ICA), fast independent component analysis (fastICA), principal component analysis, singular value decomposition, dependent component analysis, non-negative matrix factorization, low-complexity coding and decoding, stationary subspace analysis, common spatial pattern analysis and any combination thereof.

6. The system according to claim 4, wherein said circuit is configured for detecting muscle unit action potential (MUAP) activity based on an output of said blind source separation algorithm.

7. The system according to claim 1, wherein said set of electrodes comprises two subsets of electrode for receiving signals from respective two opposite sides of a portion of said skin.

8. The system according to claim 1, wherein said set of electrodes comprises two subsets of electrode for receiving signals from respective two limbs.

9. The system according to claim 1, wherein said circuit is configured to access a database storing a library of activation patterns and associated control commands, to search said database for a database activation pattern matching said identified activation pattern, and to extract from said library control commands associated with said matched database activation pattern.

10. The system according to claim 9, wherein said circuit is configured to transmit said extracted control commands to an appliance.

11. The system according to claim 1, wherein said circuit is configured for at least one member of a group consisting of: determining muscle fatigue, performance training, for rehabilitation, for determining muscle pain and any combination thereof.

12. The system according to claim 1, wherein said circuit is configured to generate a warning if a parameter is outside at least one predetermined limit.

13. The system according to claim 12, wherein said warning is provided by a member of a group consisting of visually, audibly or tactilely and any combination thereof.

14. The system according to claim 1, in use in relation to plastic surgery, for a member of a group consisting of improvement of facial symmetry, during rehabilitation physiotherapy and any combination thereof.

15. The system according to claim 1, in use for neurorehabilitation.

16. The system according to claim 15, wherein said circuit is configured for at least one of: providing characterization of walking, providing assessment of post-stroke recovery, providing assessment of post-spinal cord injury motor recovery, providing spasticity assessment, providing biofeedback, employing serious games, providing indication of muscle synergies, controlling a prosthesis, controlling an exoskeleton, controlling a robot.

17. The system according to claim 1, in use for at least one of: extracting neural control strategies, myoelectric manifestations of muscle fatigue, and myoelectric manifestations of cramps.

18. The system according to claim 1, wherein said circuit is configured for identifying said locations and said activation patterns, while said subject is moving.

19. The system according to claim 1, wherein said circuit is configured for identifying said locations and said activation patterns, while said active muscles do not change their length or shape.

20. A method of determining muscle activation comprising:
adhering a set of electrodes to a skin of a subject; and
by a processor in communication with the electrodes, receiving locations of said electrodes and electrical signals detected by said electrodes, analyzing said signals to identify a section of an active muscle, identifying locations of at least segments of active muscles and activation patterns of said active muscles based on said identified section, and constructing a displayable map of said locations and said activation patterns, wherein patterns corresponding to different active muscles are distinguishable on said map.
